(19) United States
(12) Patent Application Publication
(10) Pub. No.: US 2020/0077900 A1
(43) Pub. Date: Mar. 12, 2020

(54) SYSTEM, METHOD AND COMPUTER-ACCESSIBLE MEDIUM FOR DETERMINING A MAGNETIC RESONANCE IMAGING TEMPERATURE PROFILE

(71) Applicant: The Trustees of Columbia University in the City of New York, New York, NY (US)

(72) Inventor: John R. VAUGHAN, New York, NY (US)

(21) Appl. No.: 16/470,324

(22) PCT Filed: Dec. 18, 2017
(86) PCT No.: PCT/US2017/067105

§ 371 Date: Jun. 17, 2019

Related U.S. Application Data
(60) Provisional application No. 62/498,150, filed on Dec. 16, 2016.

(51) Int. Cl. A61B 5/01; A61B 5/055; A61B 5/00

(52) U.S. Cl. CPC A61B 5/015; A61B 5/055; A61B 5/004; A61B 5/0022; A61B 5/0013

(57) ABSTRACT

An exemplary system, method and computer-accessible medium for generating a magnetic resonance imaging (MRI) temperature profiles of a portion(s) of a patient(s) can be provided, which can include, for example receiving first imaging information related to an MRI scan of the portion(s) of the patient(s), generating second imaging information by segmenting the first imaging information into a plurality of layers, and generating the MRI temperature profile(s) by applying a bioheat equation(s) to the second imaging information. The first imaging information can be based on a point-by-point MRI scan of the patient(s). The first imaging information can be a Digital Imaging and Communications in Medicine data set produced by an MRI apparatus.

### CROSS-REFERENCE TO RELATED APPLICATION(S)

This application relates to and claims priority from U.S. Patent Application No. 62/498,150, filed on Dec. 16, 2016, the entire disclosure of which is incorporated herein by reference.

### FIELD OF THE DISCLOSURE

The present disclosure relates generally to magnetic resonance imaging (“MRI”), and more specifically, to exemplary embodiments of an exemplary system, method and computer-accessible medium for determining a magnetic resonance imaging temperature profile.

### BACKGROUND INFORMATION

Penne's bioheat equation is commonly used to determine the effects of MRI on a subject. However, Pennes' Bioheat Equation is artificially limited by a constant temperature for blood perfusion.

Thus, it may be beneficial to provide exemplary system, method and computer-accessible medium for determining a magnetic resonance imaging temperature profile which can overcome at least some of the deficiencies described herein above.

### SUMMARY OF EXEMPLARY EMBODIMENTS

An exemplary system, method and computer-accessible medium for generating a MRI temperature profiles of a portion(s) of a patient(s) can be provided, which can include, for example receiving first imaging information related to an MRI scan of the portion(s) of the patient(s), generating second imaging information by segmenting the first imaging information into a plurality of layers, and generating the MRI temperature profile(s) by applying a bioheat equation(s) to the second imaging information. The first imaging information can be based on a point-by-point MRI scan of the patient(s). The first imaging information can be a Digital Imaging and Communications in Medicine data set produced by an MRI apparatus.

In some exemplary embodiments of the present disclosure, the layers can include at least three layers, where a first layer of the layers can be a high water content, a second layer of the layers can be a low water content, and a third layer of the layers can be an air content. The layers can include at least four layers, where a fourth later of the layers can be the portion(s) of a lung. A conductivity or a permittivity can be assigned to the second imaging information associated with the portion(s), where the bioheat equation(s) can be based on the conductivity or the permittivity. The conductivity or the permittivity can also be assigned based on a thermal property(ies) of the portion(s).

In certain exemplary embodiments of the present disclosure, the thermal property(ies) can include of (i) a specific absorption rate, (ii) a specific heat, (iii) a density or (iv) a perfusion. The MRI temperature profile(s) can include areas to heat and magnitude assignments for the portion(s) of the patient(s). The bioheat equation(s) can be based on a specific absorption rate of the portion(s) of the patient(s).

A further exemplary system, method and computer-accessible medium for generating a MRI of a portion(s) of a patient(s) can be provided, which can include, for example performing a first MRI scan of the portion(s) of the patient(s) using an MRI apparatus, generating first imaging information based on the first MRI scan, transmitting the first imaging information over a network, receiving, over the network, second imaging information related to a MRI temperature profile(s) of the portion(s) that can be based on a bioheat equation(s), modifying parameters of the MRI apparatus based on the second imaging information, and performing a second MRI scan of the portion(s) using the modified parameters.

In some exemplary embodiments of the present disclosure, the modified parameters can include local areas of heating and magnitude assignments for the portion(s). The first imaging information can be based on a point-by-point MRI scan of the patient(s). The bioheat equation(s) can be based on a specific absorption rate of the at least one portion.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1A and 1B are exemplary images of relative RF magnetic field and specific absorption rate generated using the exemplary system, method and computer-accessible medium according to an exemplary embodiment of the present disclosure;

FIG. 2A is an exemplary set of images obtained using an exemplary coil arrangement according to an exemplary embodiment of the present disclosure;

FIG. 2B is an exemplary diagram of a 16 channel transmit whole body coil according to an exemplary embodiment of the present disclosure;

FIG. 3 is an exemplary thermal map of a pig atlas generated using the exemplary system, method and computer-accessible medium according to an exemplary embodiment of the present disclosure;

FIG. 4 is a graph of the exemplary bioheat model compared to Penne's bioheat model according to an exemplary embodiment of the present disclosure;

FIG. 5 is a set of temperature maps of a pig generated using Penne's bioheat equation;

FIG. 6 is a set of temperature maps of a pig using the exemplary bioheat equation generated using the exemplary system, method and computer-accessible medium according to an exemplary embodiment of the present disclosure;

FIG. 7 is an exemplary graph of the exemplary temperature map prediction with a core temperature measurement thermometer generated using the exemplary system, method and computer-accessible medium according to an exemplary embodiment of the present disclosure;

FIG. 8A is an exemplary image of a thermal hotspot generated using the exemplary system, method and computer-accessible medium according to an exemplary embodiment of the present disclosure;

FIG. 8B is an exemplary graph of the measured thermal hotspot generated using the exemplary system, method and computer-accessible medium according to an exemplary embodiment of the present disclosure;

FIG. 9 is an exemplary image of a RF heating apparatus according to an exemplary embodiment of the present disclosure;

FIG. 10 is an exemplary block diagram of a RF heating apparatus according to an exemplary embodiment of the present disclosure;

FIG. 11 is a set of images of a 16 channel TEM clamshells body coil according to an exemplary embodiment of the present disclosure;

FIG. 12 is an exemplary flow diagram of a pre-scan RF safety protocol according to an exemplary embodiment of the present disclosure;

FIG. 13 is an exemplary set of thermal maps of RF shim settings for the exemplary 16 channel TEM coil according to an exemplary embodiment of the present disclosure;

FIG. 14 is an exemplary set of thermal maps comparing 7 T to 10.5 T for FR field, loss and temperature predictions generated using the exemplary system, method and computer-accessible medium according to an exemplary embodiment of the present disclosure;

FIG. 15A is an exemplary image of a pig model according to an exemplary embodiment of the present disclosure;

FIG. 15B is an exemplary graph of the GBHT model according to an exemplary embodiment of the present disclosure;

FIG. 16 is a set of images of a passive magnetic shield and a copper Faraday cage;

FIG. 17A is an exemplary flow diagram of an exemplary method for generating a magnetic resonance imaging temperature profile of a portion of a patient according to an exemplary embodiment of the present disclosure;

FIG. 17B is an exemplary flow diagram of the exemplary method for generating a magnetic resonance imaging temperature profile of the portion of the patient according to another exemplary embodiment of the present disclosure; and

FIG. 18 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The exemplary system, method and computer-accessible medium, according to an exemplary embodiment of the present disclosure, can utilize an exemplary mechanistic bioheat transfer model to replace the empirical Pennes bioheat equation. An exemplary model can be translated into a pre-scan RF safety protocol for full clinical utility. Thus, the exemplary system, method and computer-accessible medium, according to an exemplary embodiment of the present disclosure, can replace standard model specific absorption rate (“SAR”) calculations with patient specific temperature predictions for safety assurance and protocol planning. This can be performed, for example, by rapidly imaging human subjects from head-to-toe, segmenting their anatomy, assigning electrical and thermal constants to these anatomic segments and the calculating SAR and then temperature using the exemplary bioheat equation, all in a short or relatively short (e.g., five-minute) pre-scan protocol. This accurate and precise, patient specific temperature prediction, can significantly improve both RF safety and RF pulse protocol performance as compared to the SAR metric generally used today.

### Exemplary MR Scanners

The exemplary common mode rejection ratio (“CMRR”) house can include an array of high-field magnetic resonances.

### Exemplary Human Systems (Whole Body):

3 Tesla/90 cm bore, Siemens Trio console;
4 Tesla/90 cm bore, Varian/Siemens console;
7 Tesla/90 cm bore, Siemens console, 40 mT/m body gradients, 80 mT/m head gradient insert;
7 Tesla/90 cm bore, Siemens console 70 mT/m body gradients;
9.4 Tesla/65 cm bore, Varian console (human/animal system); and
10.5 Tesla/88 cm bore, Siemens console 70 mT/m body gradients (operational).

### Exemplary Small-Bore Systems:

4.7 Tesla/40 cm bore, Varian console;
9.4 Tesla/31 cm bore, Varian console; and
16.4 Tesla/26 cm bore, Varian console.

### Exemplary Multi-Channel RF Transmit Systems

All of the human systems can have multi-channel transmit capability to handle high field transmit B₁ distortions, as well as multi-channel receivers, which can facilitate the use of parallel imaging procedures that utilize simultaneous sampling of MR signals from independent probes. The current capabilities of the different systems can include, for example:
3 T: Siemens console with dual channel whole body transmit, 32 receive channels;
4 T: Varian console with 8 channels transmit and 16 channels receive;
7 T: Siemens console 16 channel CMRR designed multi-channel transmit system and 64 receiver channels;
7 T: Siemens console 16 channel CMRR designed multi-channel transmit system, plus a 16 channel Siemens parallel transmit system, and 32 receiver channels;
9.4 T/65 cm: Varian console with 16 channels transmit and 32 receive;
10.5 T/88 cm: Siemens console with 16 2 kW transmit channels and 32 receivers;
16.4 T: Varian console with 8 transmit and 8 receive channels;
9.4 T/31 cm: Varian console with 8 transmit and 8 receive channels;
10.5 T/88 cm bore: This exemplary whole body system is a high field whole body system. The exemplary system can include, e.g., a Siemens console with 16 parallel transmitters (2 kW/channel) and 32 receive channels. The whole body gradients can provide up to 70 mT/m per axis with a maximum slew rate of up to 200 T/m/s. To correct susceptibility gradients, the shim set can be augmented to include 3^{rd} order shims driven with 20 A/channel. The patient space is 65 cm in diameter, similar to most clinical systems;

| | |
|---|---|
| Field/diameter | 10.5T/880 mm; |
| Temporal stability | 0.03 ppm/hour; |
| Spatial | <0.07 ppm 250 mm dsv; |
| homogeneity | |
| Conductor | NbTi; |
| Temperature | 3 K; |
| Size | 4.1 × 3.2 m; |
| Weight | 110 tons; |
| Console | Siemens; |
| Gradients | SC72 whole body gradient (70 mT/m per axis, |
| | 200 T/m/s) with 3rd order shims 15 A/shim; |
| Transmit RF | 16 waveform generators and 16 2 kW RF amplifiers; |
| Receive RF | 32 Receive channels based on Tim RF technology; and |
| Patient Bore | 65 cm diameter. |

### Exemplary Molecular Imaging

The expanded CMRR facility can house:
Human PET/CT—Siemens Biograph mCT (64) 4-ring scanner with an integrated 64 slice scanner;
Quad Head SPECT camera—4 fixed 90 degree ring small FOV ultra high resolution detectors; and
Micro PET/CT—Siemens Inveon preclinical microPET/CT with integrated animal monitoring and isoflurane gas anesthesia system

### Exemplary Cyclotron

Current safety practices are based more on simplified models, educated guesses, conservative guidelines and “experience,” than on hard data and complete understanding. The magnetic resonance (“MR”) industry, for example, focuses on predicting safety by SAR calculations from static “standard” models of human anatomy. It is well understood, however, that thermogenic pain, damage and stress to cells, tissues and systems can be consequences of excessive temperature, and not to SAR by itself. SAR is but one of six or more parameters of bioheat equations that can be solved in order to determine temperature. SAR alone completely ignores the thermodynamics and the physiology of the safety concern. It is also well known that standard models cannot possibly be used to predict SAR or temperature for the nearly infinite number of variables presented by patient size, shape, mass, position, physiology, disease, RF coil circuit and RF imaging protocols. Thus, SAR is relied upon as the safety metric not because it accurately predicts or measures human safety, but because the more difficult problems of accurately and precisely predicting and measuring absolute temperature in the human body have not been solved. Until the magnetic reasonable imaging (“MRI”) field adapts “temperature” as its safety metric and, patients may not be as safe, imaging protocols can often be compromised. Improving upon the existing “gold standard” empirically based Pennes' Bioheat Equation that is artificially limited by a constant temperature for blood perfusion, the exemplary system, method and computer-accessible medium, according to an exemplary embodiment of the present disclosure, can include a mechanistically based “Generic Bioheat Transfer Model”. The significant improvement in the exemplary model, and temperature predictions using the exemplary model, were calibrated and validated through direct fluoroptic probe thermometry with anesthetized pigs.

### Exemplary Bioheat Model Used to Parallel Transmit Temperature Predictions

The body of the RF Safety Research to date has addressed heating from an RF field generated by a single RF transmit coil source. In recent years, however, RF fields generated by multiple transmit coil elements have become popular in clinical and research applications at 3 T and 7 T. While bringing many benefits for RF field optimization, shifting current magnitude and phase over time and space with these coils makes predicting and tracking RF heating a moving target. The exemplary system, method and computer-accessible medium, according to an exemplary embodiment of the present disclosure, can utilize the exemplary bioheat model to accurately and precisely predict RF heating in the subject for parallel transmit applications.

### Exemplary Bioheat Model-Based Protocol for Precision Temperature Predictions

It can be beneficial to predict, with high confidence of accuracy and precision, the absolute temperature contours resulting from MRI, in human subjects. The exemplary system, method and computer-accessible medium, according to an exemplary embodiment of the present disclosure, can include scanning an individual subject, segmenting the anatomy, assigning electrical properties to the segments, calculating SAR, assigning thermal properties to the anatomy and then predicting temperature contours in the anatomy by translating SAR to temperature using the exemplary extended bioheat model.

### Exemplary Animal Studies

A total of 100, human-adult-sized, 60-80 kg pigs were used over the first 4 years of the exemplary study. These animal experiments facilitated the modeling workflow to be refined and automated, the modeled relationship between SAR, static temperature, and perfused temperature to be refined and verified, and validated real-time thermal management by comparing the thermal predictions with invasive thermometry in human-sized porcine models.

Each pig was rested for at least 6 days after its arrival to the animal facility of the CMRR to avoid anxiety, and fasted for 12 hours before the induction of anesthesia to avoid complications. Water was provided ad libitum during the fasting. For the experiment, first the animal can be immobilized and sedated using 5-10 mg/kg Telazol (e.g., Tiletamine HCL+Zolzepam HCL). The animal was weighed to calculate the RF power needed for the intended whole-body average SAR exposure. This was followed by intubation or tracheotomy. The animal was kept anesthetized during the experiment (e.g., approximately 6 hours) using 2-3% isoflurane in 50%-50% air-O2. Respiratory rate was set to 12-13 cycles/min using a ventilator (e.g., Ohmeda 7000). Minute volume was set between 7-8 L/min. Saline (e.g., 0.9% NaCl) was provided through an ear vein at the rate of approximately 0.4-0.6 L/hour to keep the animal hydrated during the experiment.

The animal was placed in the MR scanners and whole-body fat/water scans were acquired to generate an animal-specific 4-tissue type body mesh for EM simulation, SAR calculation, and thermal prediction. The animal was returned to the RF safety lab for controlled RF heating experiments. RF heating was measured invasively using 16 fluoroptic temperature probes. The temperature probes were surgically placed at the locations within the pig predicted by thermal modeling to be regions for the RF phase setting (e.g. driving the coil in a circularly polarized manner). These locations included “hot spots”, as well locations within strong thermal gradients. One probe each was placed in the rectum and air near the animal in the coil. The room temperature and humidity were recorded. Temperatures were recorded before the deposition of RF power (e.g., pre-RF epoch), during the RF power deposition (e.g., RF epoch), and after the termination of the RF power (e.g., post-RF epoch). Net RF power (e.g., forward-reverse) delivered to the body coil was measured in the coaxial cable leading to the coil using a power meter. At the end of the experiment, the animal was euthanized using a saturated pharmaceutical grade KCl solution. An additional non-perfused data set using was acquired to compare perfused vs. non-perfused local heating.

### Exemplary Justification for the Porcine Model of Heating

Pigs can be used to study RF heating since pig is a thermo-physiologically similar and conservative animal model of a human. A pig has human-comparable thermal mass, surface area, water loss through skin below its critical, hot environmental temperature, metabolic energy per unit surface area, cardiac output, electromagnetic and thermal properties, and thermoregulatory mechanisms—making it thermo-physiologically similar to a human. The critical hot environmental temperature limit for a pig (e.g., 36° C. for a newborn, 30° C. for a mature) is comparable to and lower than that of a human (e.g., 37° C. for a newborn, 43° C. for a mature)—making it a thermo-physiologically conservative animal model of a human. Human comparable thermal mass, surface area and electromagnetic properties facilitate a pig to load a body coil similar to a human.

### Exemplary Protection of Human Subjects

Exemplary vitals were measured including: height, weight, heart rate, core temperature and skin temperature. The volunteer was outfitted with physiologic monitoring equipment, fiber-optic thermal probes and a communications squeeze ball. RF coils were placed on the volunteer and then they were rolled into the magnet. Using the methods that were automated and refined during the preceding porcine studies, a whole-body, 3-echo time Dixon imaging scout scan was collected. That data was distilled into patient-specific fat and water maps. From this, and some geometric information, the voxels in the body were classified as one of 4 tissue types: (i) high water content, high conductivity tissues (e.g., muscle, brain, organs), (ii) low water content, low conductivity (e.g., fat and bone), (iii) internal air (e.g., trachea, lungs), (iv) and skin. This exemplary model of the person was placed in a pre-designed RF coil mesh, and FDTD electromagnetic simulations were computed on an array of GPUs to calculate the per-channel Electric- and Magnetic-fields generated by each coil. The fields, the electrical properties and tissue density of the personalized mesh, the relative transmit phases and magnitudes, and the energy content of the protocol was combined for each experimental protocol to keep a running calculation of voxel by voxel SAR. This information served as input to the generic bioheat equation thermal solver, which calculated the temperature at each point in time, including imaging scans that were queued, but not yet acquired. While this series of calculations were completing, standard image planning and scanner adjustments can continue. Such scans can generally not be RF intense and because RF heating is the summation of all RF scans, at the beginning of a scanning session RF heating is far from thermal limits. Once the calculations were completed, all scans to that point in time were summed and the exemplary thermal model was determined. The operator had real-time feedback of thermal hot spots and the temperature matrix was monitored so that FDA thermal limits were not exceeded. Staying within SAR guidelines, several minutes of both Turbo Spin Echo and balanced Steady State Free Precession imaging were acquired on each volunteer.

### Exemplary Description of Potential Risks and Discomforts

There are four parameters that can be considered as potential risks in an MR study: (i) static magnetic field strength, (ii) rate of change of magnetic field (e.g., dB/dt), (iii) RF power deposition and (iv) acoustic noise level. Parameters (i), (ii) and (iv) may present any significant risk to subjects. Parameter (i) may not present a significant risk at 3 T and 7 T, these fields being below FDA “non-significant risk” field strength of 8 T. Parameter (i), dB/dt can be the same at the scanners, and may not pose a significant risk since they can be kept below identified thresholds. Risks from parameter (iv) can be routinely mitigated with pulse sequence adjustment and hearing protection at 3 T and 7 T; the same can be expected at 10.5 T. Parameter (iii) can be restricted to the FDA allowable maximum whole-body average SAR of 4 W/kg. More accurate RF risk assessment and reduction can be the beneficial to determine. Reducing this risk at or close to the minimum by keeping maximum temperatures below the FDA temperature thresholds can be beneficial.

### Exemplary Risk Protecting Procedure

A number of exemplary procedures can be taken to protect against potential risks in addition to keeping dB/dt and acoustic noise levels within the FDA guidelines. During each study, the subjects were continuously monitored visually and communicated with the researcher immediately, and were removed from the magnet if needed. In addition to this, subjects also had access to a “panic” alarm in the magnet which notified the investigators of a problem and the desire to immediately stop the study. RF power deposition from a coil was monitored with exemplary software and hardware protection systems, and was kept below the specified maximum limit for an experiment. RF power deposition duration was determined using the exemplary bioheat model such that the maximum body temperature was kept below the FDA temperature thresholds.

The potential risk of magnetic objects being attracted by the magnet can be minimized. Additionally, exemplary procedures can be used to avoid the presence of ferromagnetic objects in the magnet room. Subjects can experience dizziness while being moved in and out of the magnet, the severity of which can be proportional to the speed with which the table moves, and thus can be minimized by moving the subject in and out of the magnet slowly. Acoustic noise can be reduced by a shield placed inside the gradient, by the use of specialty ear plugs (e.g., Howard Leight Industries, San Diego, Calif.) which can reduce the acoustic noise by 33 dB, and by acoustic foam padding. At 10.5 T, other possible effects were determined by administering exit questionnaires to all subjects.

Healthy humans and patients were regularly imaged in 3 T and 7 T MR scanners at the whole-body average SAR of 4 W/kg (e.g., the maximum allowable SAR in the first level controlled mode) without considerations for the in-vivo temperatures and imaging time. The exemplary system, method and computer-accessible medium, according to an exemplary embodiment of the present disclosure, can be used to investigate, better understand, predict, validate and measure temperature to better assure safety at 3 T, 7 T, and ultimately at 10.5 T, risks to subjects can be significantly less than risks considered reasonable on existing FDA approved commercial systems in clinics and research labs. An exemplary bioheat model and MR thermometry method, and in vivo RF heating data in human-adult-sized pigs, can help significantly reduce the risks by keeping maximum body temperatures reached below FDA thresholds.

RF heating in ultra-high field MRI is not well understood. (See, e.g., Reference 1). The consequences of not knowing the temperature induced by a RF excitation protocol in a human body can be two-fold: (i) Human safety is based on a “guess” and (ii) a “safe” guess needs the use of overly conservative, low-performance, pulse protocols. A better understanding of RF heating can make high-field MRI significantly safer and more powerful.

A significant problem can be that modern high-field MRI systems and practice rely on predicting and monitoring SAR for safety compliance. (See, e.g., References 2, 3, 4, 5, 6, 7 and 8). This approach can be wrong, however, being both protocol limiting and potentially dangerous. The SAR approach to RF safety is taken because RF power input into a coil can be easy to measure. At lower field strengths (e.g., <1.5 T) and longer Larmor wavelengths (e.g., >50 cm in tissue), SAR distribution over a body can be more uniform. Average SAR input in units of average watts per kilogram can be easy to conceptualize. SAR's effect on heating is also more uniform. At these lower fields, less SAR can be expended to make an image by a standard pulse sequence; there can be less tissue loss and therefore less heating. “Being conservative” with RF power use was “good enough” for safe and successful MRI. Problems can arise with this “averaged SAR” approach as MRI is advanced into higher fields in search of higher SNR and other benefits. As the Larmor wavelengths in high-water content tissue decrease to about 28 cm, about 12 cm, and about 8 cm for 3 T, 7 T, and 10.5 T respectively, RF excitation wavelengths can become significantly smaller than anatomic dimensions, leading to highly non-uniform B1 and E fields over the body. When this excitation signal comes from multiple coil element sources as with a body coil, interference patterns can further degrade RF field uniformity into highly localized patterns of excitation B1 field, SAR, and temperature. These local patterns of B1, SAR and T may not spatially correlate. As RF tissue loses, both ohmic and dielectric, increase in respective direct and quadratic proportion to frequency in SAR increases as well. To acquire a given image, more power can be needed which can result in more SAR and heating. The need therefore to better understand, predict, and measure this heating becomes critical to the effectiveness of the MR application, and most importantly to the safety of the human patient or research subject. It can be equally important to understand that temperature T, and not SAR, can be the source of sensation (e.g., pain), thermogenic cellular damage (e.g., burns) and systemic stress (e.g., heat stroke). Measuring SAR accounts for the electrodynamics only, and not the thermodynamics or thermoregulatory response of the living system under study. SAR can be but one of six parameters utilized to calculate temperature in the bioheat equations and by itself an inadequate predictor or indicator of safety. The exemplary system, method and computer-accessible medium, according to an exemplary embodiment of the present disclosure, in contrast to only using SAR, can predict and measure the absolute temperature magnitude with sufficient accuracy and precision (e.g., 0.2° C.) to facilitate careful protocol planning and safety assurance. At higher fields, the safety margins can be too close to continue with the inertia of practicing safety the old way.

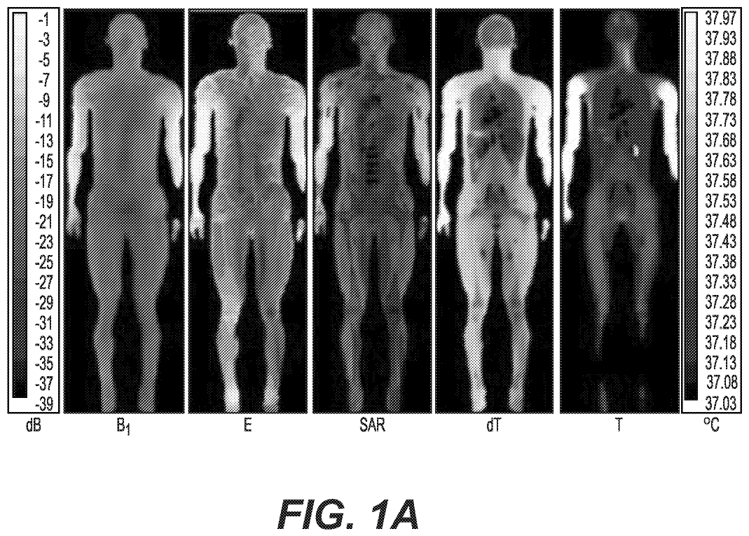

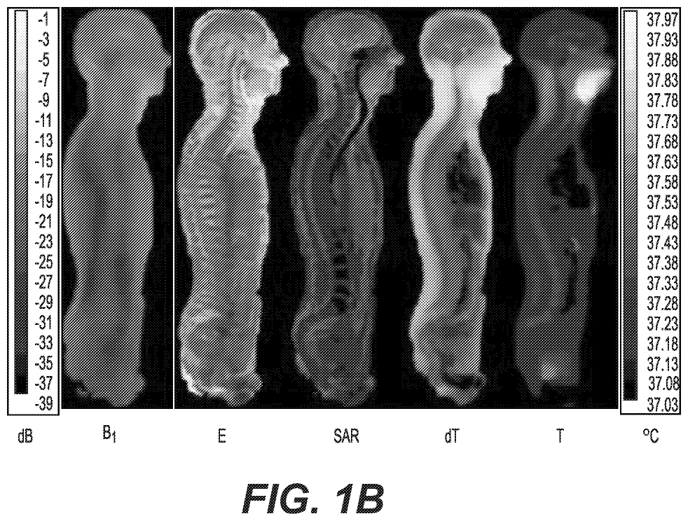

FIGS. 1A and 1B show exemplary thermal images of relative RF magnetic field and specific absorption rate generated using an exemplary system, method and computer-accessible medium according to an exemplary embodiment of the present disclosure. For example, FIG. 1A shows sagittal views and FIG. 1B shows coronal views of the whole body inside a transmitting body coil within the shielded magnet bore SEMCAD (e.g., SPEAG, Zurich). Finite difference time domain methods were used to numerically solve electrodynamic and thermodynamic parametric contours used for understanding of RF propagation, loss and consequential heating in the body at ultra-high frequencies. SAR shows a highly non uniform distribution, concentrated primarily in the arms and tissues peripheral to the body core nearer to the coil elements as expected, but can be counter-intuitively high well outside of the body coil's elements, in the legs and especially in the head and brain. This underscores an important understanding that SAR must be considered for the whole body and not simply the trunk within the active elements field of view (“FOV”) of the coil. The heating increase (e.g., dT) correlates approximately spatially with the SAR distribution as might be expected, except that the well perfused brain can be cooler than SAR contours alone would predict. However, the heating change may still not be as important as the absolute temperature magnitude obtained. The temperature contours predicting hot arms, shoulders, neck, and crotch. Indicative of the difficulty of predicting understanding heating in the anatomy, a single kidney is shown to be >1.0° C. The upper legs can be warm. Interestingly the portion of the trunk within the coil remains quite cool. As shown in the FIGS. 1A and 1B, RF energy is input at 1.8 W/kg, well under the 4 W/kg SAR guideline limit for the body. However the arms and shoulders, neck and the kidney have reached or exceeded the guideline temperature limit in the head and body. (See, e.g., References 1 and 9). The temperature calculated above by Pennes' Bioheat equation can be improved by including dynamic temporal and spatial blood temperature used in the exemplary bioheat model. The models of FIGS. 1A and 1B illustrate clearly demonstrate that the imaging volume (e.g., B1), SAR, temperature change (e.g., dT), and absolute temperature magnitude (e.g., T) can be independent parameters. None can be substituted for, or used to predict, temperature T, apart from their relationships through the bioheat equations.

In addition to the general need for understanding, predicting, and measuring high field MRI induced heating as described above, a number of practical needs can be pressing for these solutions as well. While human patients and healthy subjects can be imaged at 3 T-10.5 T, RF heating data in-vivo at these ultra-high fields can be scarce to nonexistent. (See, e.g., References 10, 11, 12, 13, 14, 15 and 16). No RF heating data in-vivo is found in the literature for whole body imaging at 3 T and above. This can be alarming considering that more than 2000, 3 T clinical systems and 40, 7 T research systems currently exist. All “clinical” 3 T and 7 T systems in use today have FDA clearances based primarily on SAR models as shown in FIGS. 1A and 1B.

The exemplary bioheat transfer model, and its use as a fast and accurate thermal predictor map validated in human-adult-sized porcine models, can address a major future research need as stated by the International Commission on Non-Ionizing Radiation Protection (“ICNIRP”): “further investigation to define more precisely the spatial deposition of RF energy during an MR procedure and the corresponding temperature fields in the human body using a three dimensional bioheat transfer model”. (See, e.g., Reference 1).

An exemplary goal can be (i) to accurately image a specific subject head-to-toe in 3D, (ii) to segment the image, to assign electrical and thermal properties to the segments, (iii) to accurately calculate the resultant absolute temperature contours expected from a given RF protocol and (iv) to generate thermal “maps” which can be used to predict and plan safe and effective, subject specific MR scans. This sequence of tasks can be performed in about a five-minute, pre-scan “RF Safety Protocol”.

### Exemplary 3d Image and Heating Data Acquisition

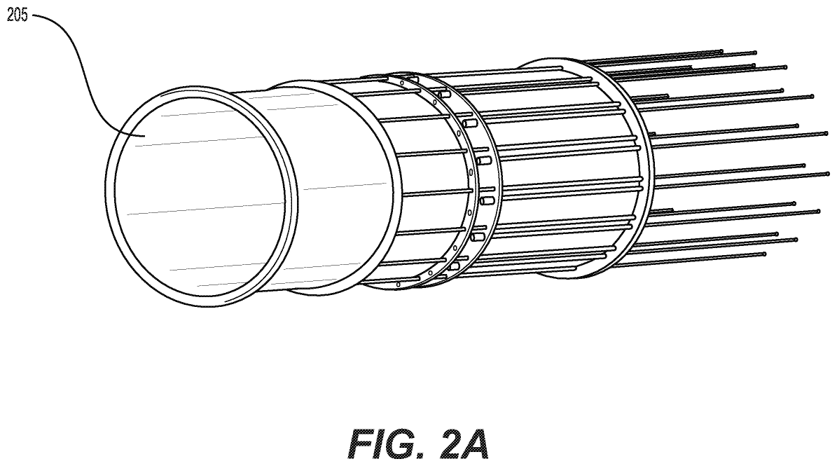

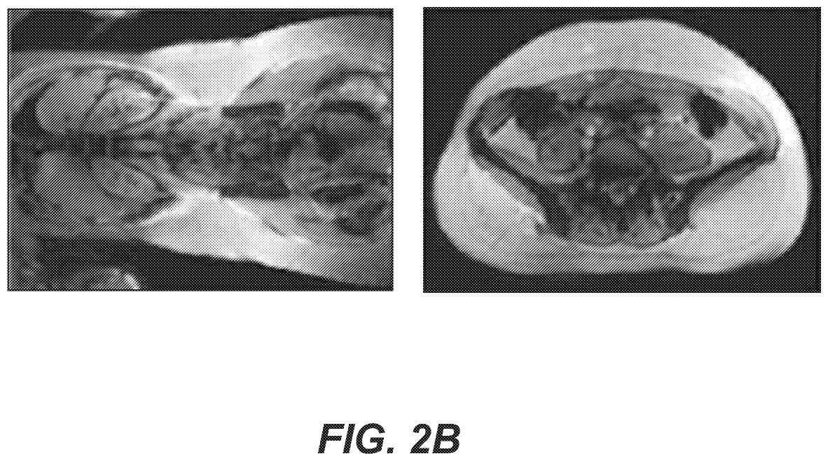

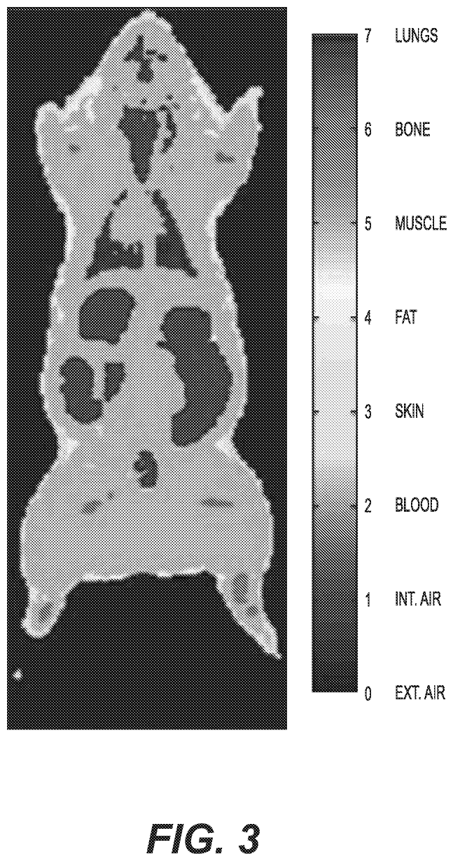

In order to measure and predict RF heating in live pigs and humans by MRI, a series if body coils were built for 3 T, 7 T and 10.5 T. While whole body coils for 3 T and 7 T currently exist, (see, e.g., References 17 and 18) new body coils were built to interface with the new Siemens Magnetom 7 T as well as a whole body coil at 10.5 T. A preliminary 16 transmit channel coil together with element decoupling and B₁ shimming was used for whole body coil imaging, demonstrating whole body human images from 7 T. (See e.g., diagram shown in FIG. 3). 7 T simulation results from this coil are seen in FIGS. 1A and 1B. Image results are shown in the images of FIG. 2B. These coils can first be used in the magnet to image pig and human anatomy for subject specific thermal map calculations. They can then be used in the RF Safety Lab to validate the exemplary calculated predictions in pig models by the approach described in the next section of this proposal. FIG. 2A shows a 16-channel transmit body coil 205 (e.g., by TEM or dipole design).

### Exemplary Anatomic Atlas Generation

From 3D images acquired from specific subjects, the anatomy can be segmented into at least four segments: (i) high water, high conductivity tissue, (ii) low water, low conductivity tissue, (iii) air and (iv) skin. These segments can be assigned specific electrical properties (e.g., conductivity, permittivity and permeability), thermal properties (e.g., specific heat, thermal conductivity, density, vascular bed density, perfusion rate and metabolic heating) and any additional factors such as physiological factors including heart rate, respiratory rate, environmental temperature, patient clothing or insulation, air flow and any conditions that might compromise thermoregulatory reflexes like sedation or disease states. Subject mass and position within the coil can be noted. The sum total of this information can be used to generate a physiological, anatomic atlas for a specific subject and scan protocol.

### Exemplary Validating the Exemplary Bioheat Transfer Model

Accurate anatomy must be accompanied by an accurate thermal solver to produce and accurate temperature map. The exemplary bioheat model can be used to predict the RF energy thermal transport and RF heating during whole-body MRI in ultra-high fields. (See, e.g., Reference 19). This mechanistically derived model can include a dynamic blood temperature term making it significantly more accurate than the empirically derived Pennes model, which holds blood temperature to a constant.

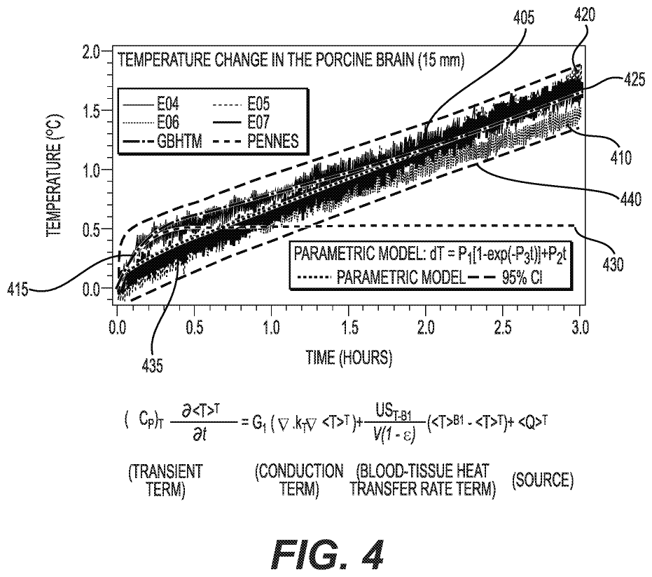

FIG. 4 shows a graph of the exemplary bioheat model compared to Penne's bioheat model according to an exemplary embodiment of the present disclosure. For example, as shown in FIG. 4, temperature change in a porcine brain can be seen for E04 (element 405), E06 (element 410), generic bioheat transfer model (“GBHTM”) thermal solver (element 415), E05 (element 420), E07 (element 425) and Pennes bioheat equation (element 430), as well as for the exemplary parametric model (element 435) and for 95% CL (element 440).

### Exemplary Temperature Maps

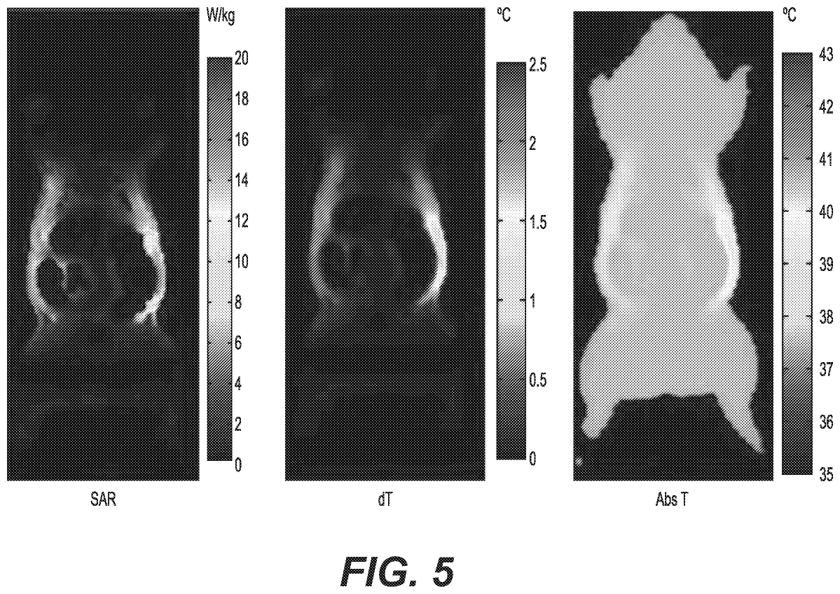

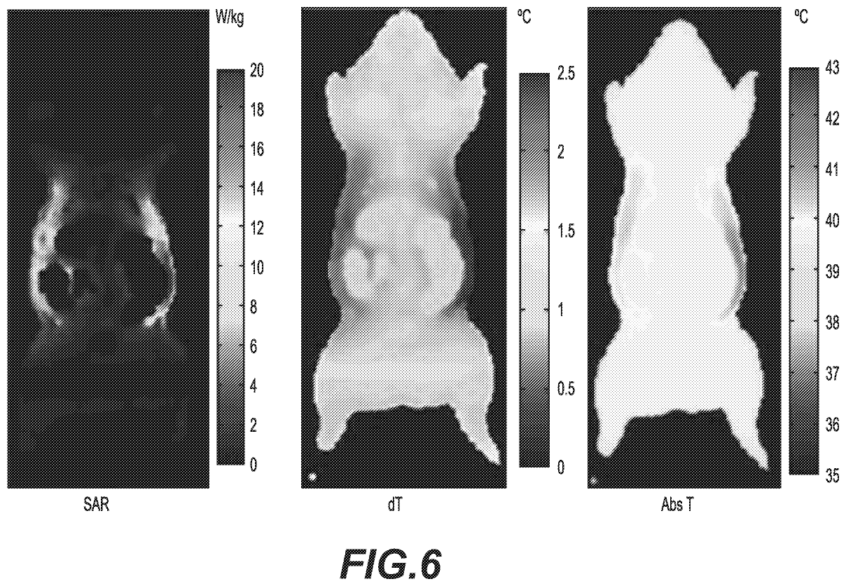

Equipped with subject specific anatomic atlas, and a new highly accurate, high precision, thoroughly validated bioheat transfer model, the SAR for a given RF coil circuit and pulse protocol can be determined, from which temperature can be calculated. For comparison, the Pennes heating rate and absolute temperature map predictions for the exemplary live pig subject is shown in the set of temperature maps in FIG. 5, and the same predictions with the same RF coil and pulse protocol, although using the exemplary new bioheat transfer model, is shown in the set of temperature maps in FIG. 6.

### Exemplary Validation

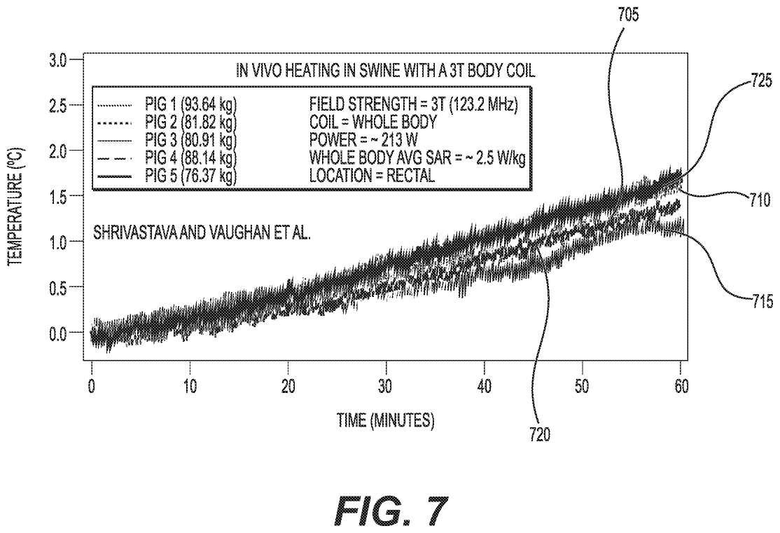

The exemplary model shown in FIG. 6, which shows a pig inside a whole body transmit coil, can be checked by measuring core temperature with a rectal thermometer. FIG. 7 shows exemplary results achieved from the exemplary model of FIG. 6 for Pig 1 (element 705), Pig 2 (element 710), Pig 3 (element 715), Pig 4 (element 720) and Pig 5 (element 725). This can demonstrate the accuracy and precision of the exemplary thermal predictions as compared to those from a commercial solver using the standard Pennes' bioheat equation.

Previously, the RF Safety Research to date has addressed heating from an RF field generated by single RF transmit coil source. However, RF fields generated by multiple transmit coil elements have become popular in clinical and research applications at 3 T and 7 T. Multi-channel transmit can also be needed at 10.5 T. While bringing many benefits for RF field optimization, shifting current magnitude and phase over time and space with these coils makes predicting and tracking SAR and RF heating a moving target. The exemplary bioheat model can be used to accurately and precisely predict RF heating in the subject for parallel transmit applications.

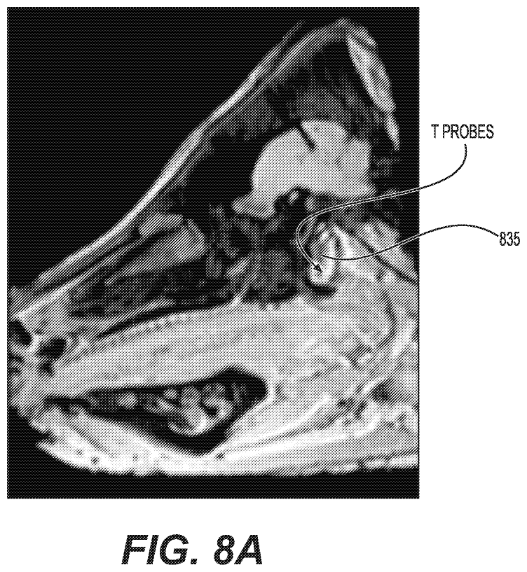

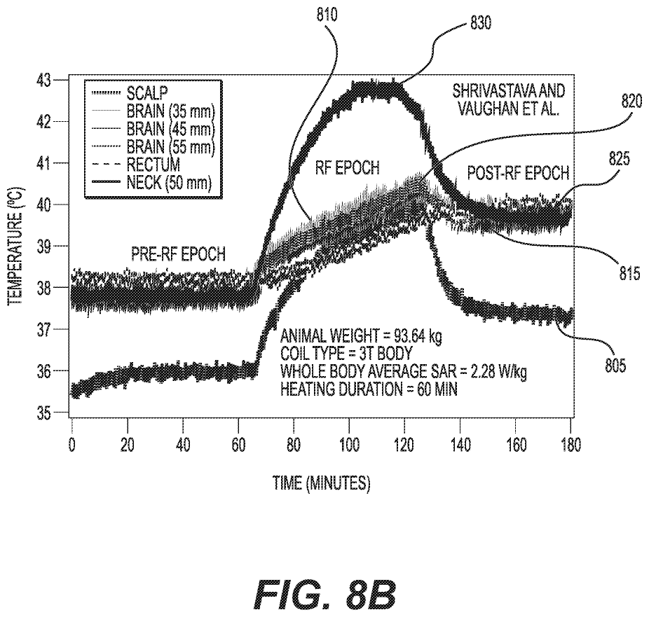

FIG. 8A shows an exemplary image of a thermal hotspot 835 generated using exemplary system, method and computer-accessible medium according to an exemplary embodiment of the present disclosure. FIG. 8B illustrates an exemplary graph of the measured thermal hotspot 835 generated for the Scalp (element 805), Brain 35 mm (element 810), Brain 45 mm (element 815), Brain 55 mm (element 820), Rectum (element 825) and Neck 50 mm (element 830) using the exemplary system, method and computer-accessible medium, according to an exemplary embodiment of the present disclosure.

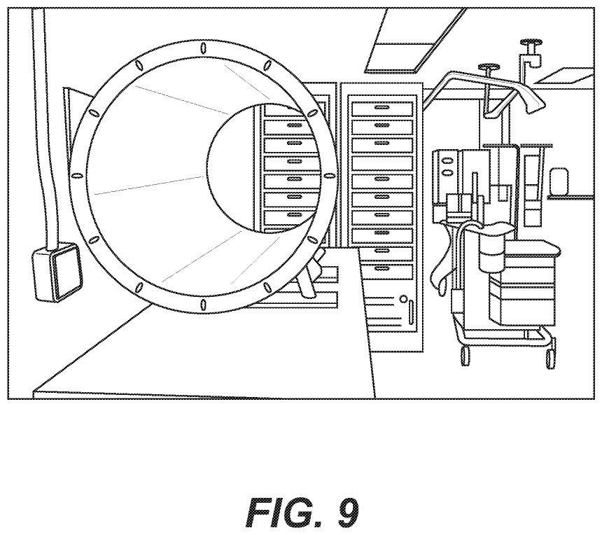

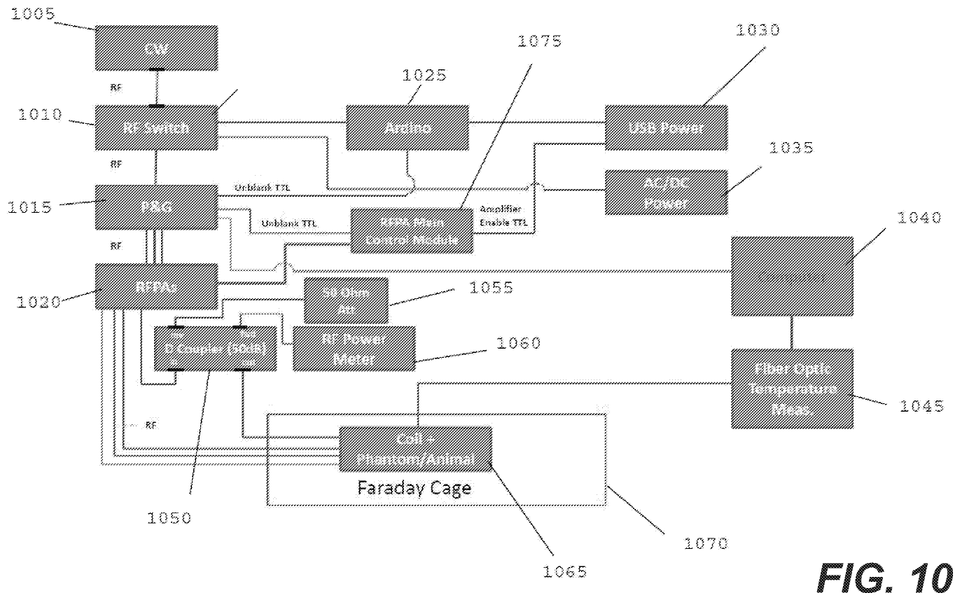

To test the ability of the exemplary bioheat thermal solver to accurately predict temperature contours resulting from different RF field contours generated by driving the exemplary multi-channel coil, the exemplary model predictions can be validated. This was performed using a RF Safety Laboratory. This included the instrumentation utilized to transmit over 16 independent channels, and to prep and support anesthetized pigs for fluoroptic probe temperature measurements. This included a 16 channel RF body coil (see e.g., FIGS. 9 and 10), tuned alternately to 127 MHz (e.g., 3 T), 300 MHz (e.g., 7 T), and 450 MHz (e.g., 10.5 T). For example, FIG. 9 shows an exemplary image of a RF heating apparatus and FIG. 10 shows an exemplary block diagram of a RF heating apparatus, according to an exemplary embodiment of the present disclosure.

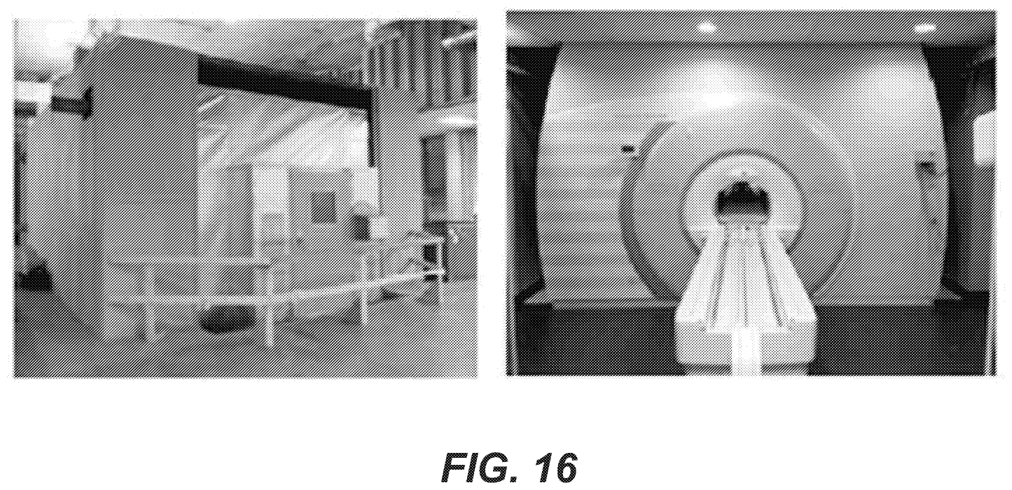

As illustrated in FIG. 10, a continuous wave (“CW”) signal generator can generate a signal and send it to a RF Switch 1010. RF Switch 1010 can be controlled by an Ardino 1025, which can be powered by a USB Power source 1030. RF Switch 1010 can be connected to P&G 1015 (e.g., be powered by AC/DC Power source 1035), which can be connected to one or more radio frequency power amplifiers (“RFPA(s)”) 1020. RFPA(s) 1020 can be controlled by a RFPA Main Control Module 1075, which can also be powered by USB Power source 1030. RFPA(s) 1020 can be connected to a Decoupler 1050 (e.g., a 50 dB decoupler) which can have a 50 ohm Attenuator 1055 and a RF Power Meter 1060 connected thereto. P&G 1015 can be controlled using a Computer 1040, which can be connected to a Fiber Optic Temperature Measurement device 1045. RFPA(s) 1020, Decoupler 1050 and Fiber Optic Temperature Measurement device 1045 can be connected to a Coil Apparatus 1065, which can be used to provide a RF signal to a Phantom/Animal. Coil Apparatus 1065 can be housed in a Faraday Cage 1070, which can be used to block outside RF signals and prevent interference by other RF signals. (See e.g., set of images of a passive magnetic shield and a copper Faraday cage shown in FIG. 16).

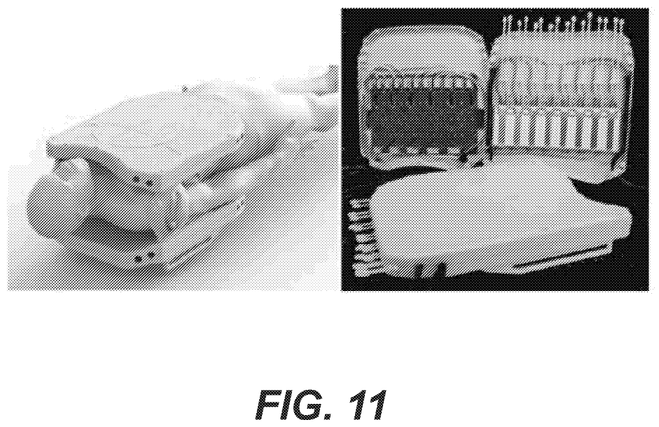

FIG. 11 shows a set of images of an exemplary 16 channel clamshell coil. The whole body coil can be used in the body transmitter in all clinical systems. The exemplary clam shell can be significantly more efficient than the whole body coil at ultra-high fields. Additionally, the entire high power RF front end of this system can be housed in an RF shielded magnet bay to comply with FCC regulations.

### Exemplary Bioheat Model Based Protocol for Precision Temperature Predictions

The exemplary system, method and computer-accessible medium, according to an exemplary embodiment of the present disclosure, can predict, with high confidence of accuracy and precision, the absolute temperature contours resulting from MRI, in human subjects. This can be accomplished by scanning an individual subject, segmenting the anatomy, assigning electrical properties to the segments, calculating the SAR, assigning thermal properties to the anatomy, and then predicting temperature contours in the anatomy by equating SAR to temperature using the exemplary extended bioheat model.

The first procedure can be to determine the size and composition of the patient, as well as their relative position in the coil. A whole body chemical shift encoded (e.g., 3 echo-time Dixon) MR scan can be performed in segments by positioning the table at a half dozen stations for scan acquisition, followed by merging the segments together. An exemplary Continuously Moving Table (“CMT”) EPI method can be implemented to reduce fat/water scout imaging to a single two minute scan. (See, e.g., Reference 23). Each voxel can be assigned fat and water fractions. (See, e.g., References 24 and 25). A four-tissue body mesh from the geometry and chemical shift information collected can be constructed with the following tissues: (i) high water content/high conductivity tissue (e.g., muscle, brain, organs), (ii) low water content/low conductivity tissues (e.g., fat and bone), (iii) internal air and (iv) skin. Knowing the geometry and proportion of these reduced tissues types can produce a more accurate SAR estimation in critical locations using an exemplary model with many tissue types (e.g., many of which can be electromagnetically similar).

The personalized biological mesh was then positioned into the pre-constructed mesh of the coil to form a merged mesh of a loaded coil. For coils using variable tune and match circuitry to accommodate different loading conditions, the virtual coil was tuned and matched using co-simulation.

Electromagnetic simulation of the loaded and tuned coil was then performed using full wave FDTD method to calculate, or otherwise determine, the steady state electric field per channel at the proton Larmor frequency. The result was the E-fields and B-fields of each channel. The SAR can be calculated in post-processing based on the E-fields, tissue parameters (e.g., density and conductivity) from the biological mesh, and transmit information (e.g., phase, magnitude, waveforms, duty cycle, duration) of each sequence. Next, the SAR was used as input to the GBHTM Thermal Solver along with the biologic mesh to predict RF heating temperature contours, and track those contours through time retrospectively from the start of the experiment on through to the queued experiments. This provided the operator with direct feedback on the most important safety concern, temperature.

### Exemplary Translation of the Exemplary Bioheat Model to Clinical Application in a Pre-Scan RF Safety Protocol

The exemplary system, method and computer-accessible medium, according to an exemplary embodiment of the present disclosure can achieve a pre-scan RF safety protocol to predict temperature contours in a specific human patient, for specific diagnostic exam protocols planned, in a clinically accepted period of time. This prediction can provide feedback utilized to tailor specific examination parameters for the optimal risk/benefit ratio for the patient.

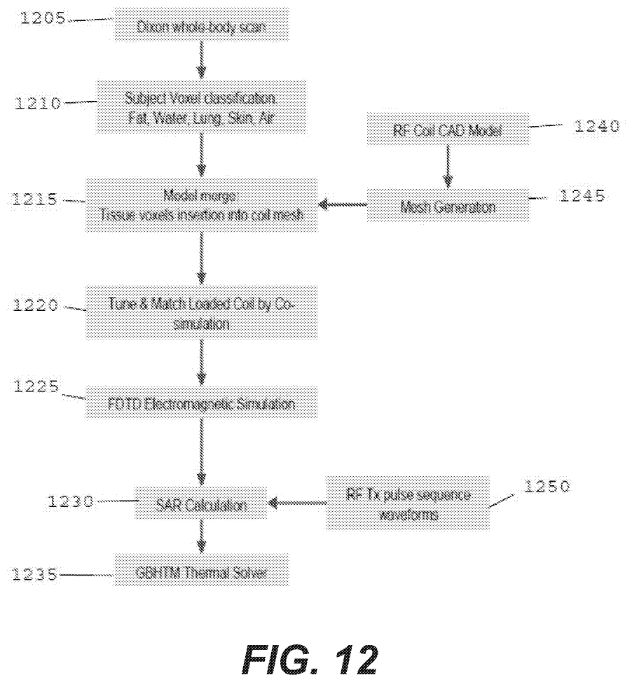

The exemplary pre-scan protocol was tested an analyzed on anesthetized pig models as shown in Table 1 below. At the rate of 15 pigs every eight months (e.g., approximately 1 pig study every two weeks) a temperature map was calculated for each pig as shown in the exemplary flow diagram illustrated in FIG. 12.

For example, as shown in the flow diagram in FIG. 12, a Dixon whole body scan can begin at procedure 1205. At procedure 1210, a subject voxel classification can be performed (e.g., classifying as fat, water, lung, skin or air). At procedure 1215, a model merge can be performed, which can include tissue voxels insertion into a coil mesh. This can be performed using a mesh generation 1245, which can be based on a RF coil CAD model. At procedure 1220, a loaded coil can be tuned and matched (e.g., using a co-simulation). At procedure 1225, an FDTD electromagnetic simulation can be performed. At procedure 1230 a SAR calculation can be performed using, for example, RF Tx pulse sequence waveforms. At procedure 1235, a GBHTM thermal solver can be generated.

To reach the minimum needed confidence levels, five pigs were imaged for each of two multi-channel body coil configurations, shimmed to each of three RF field profiles. These exemplary studies were repeated at 3 T, 7 T and 10.5 T. There were six control animals, and 4 spares, for a total of 100 pig studies over the course of 4 years.

TABLE 1
Pig Studies

| | | | | | Clamshell Coil | | |
|---|---|---|---|---|---|---|---|
| | Field | Whole Body Coil | | | | | B₁ |
| Months | Strength | Linear | Circular | B₁ shim | Linear | Circular | shim |
| 8 | 3T | 5 | 5 | 5 | | | |
| 16 | 3T | | | | 5 | 5 | 5 |
| 24 | 7T | 5 | 5 | 5 | | | |
| 32 | 7T | | | | 5 | 5 | 5 |
| 40 | 10.5T | 5 | 5 | 5 | | | |
| 48 | 10.5T | | | | 5 | 5 | 5 |

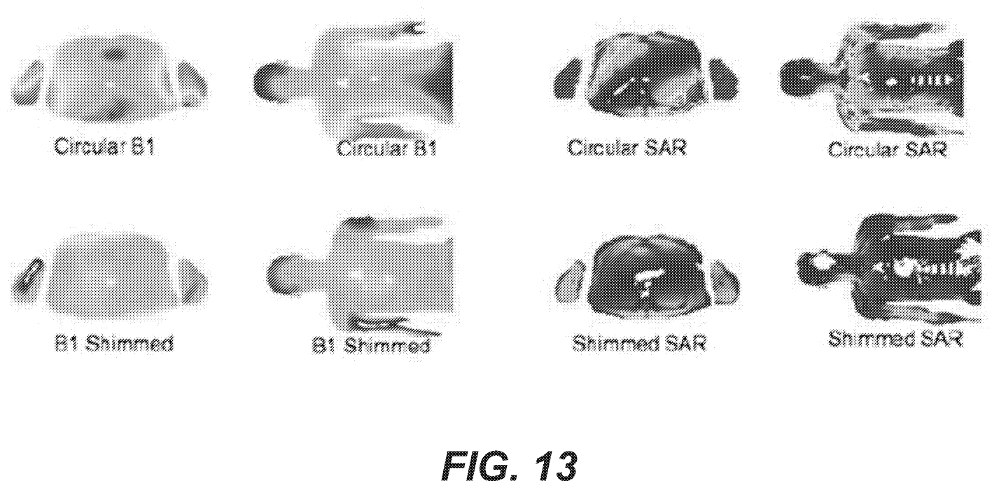

After an image set was acquired and temperature maps calculated, these animals together with the coils used were moved to the RF safety lab where the temperature predictions were directly validated by invasive temperature measurement. The temperature predictions were used to guide the temperature probe placement—in regions of maximum heating. With assumed good agreement between predictions and measurements for these multiple coil, field strength and multi-channel B1 shim conditions, the FIG. 12 pre-scan safety protocol were run on human subjects to fill in Table 2 below. FIG. 13 shows an exemplary set of thermal maps of RF shim settings for the 16 channel TEM coil according to an exemplary embodiment of the present disclosure.

TABLE 2
Plan for human studies

| Year | Field Strength | Whole body | Clam shell |
|---|---|---|---|
| 4.5 | 3T | 3 | 3 |
| 5 | 7T | 3 | 3 |
| 5.5 | 10.5T | 3 | 3 |

A challenge can be to reduce the speed of the thermal contour calculation and mapping from human subject images. However, a pre-scan safety protocol can be performed in a clinically acceptable period of time. This protocol can flag any potentially dangerous hot spots, and can convey if additional RF power can be beneficial and/or used. By utilizing temperature predictions for RF pulse protocols, RF transmit power with more flexibility, and more safety can be used.

Due to shorter wavelength attenuation at higher magnetic field strengths and Larmor frequencies, an increased amount of RF power may be needed to excite NMR spins in the anatomy that can be observed through MRI, spectroscopy (“MRS”) and functional MRI (“MRI”). (See, e.g., References 22, 27, 28, 29, 30, 35, 36, 43, and 44).

The exemplary system, method and computer-accessible medium, according to an exemplary embodiment of the present disclosure, can be used to model thermal contour prediction in the body. The exemplary system can be extended to multi-channel transmit modeling, prediction and measurement.

An exemplary porcine model was been developed and used for high clinical (e.g., 3 T) field strength validation as listed above. (See, e.g., References 22 and 43). The porcine model was used to validate models at 7 T and 10.5 T. Accurate 10.5 T models are a prerequisite for IRB and IDE clearance for human studies at this unprecedented new field strength.

MR thermometry can be used for noninvasive/minimally invasive thermal imaging in porcine model. The proton resonance shift approach was successfully employed to measure the 0.2° C. precision without calibrating against an invasive fluoroptic probe measurement. So, PRF is giving good “relative” measurement, but must be calibrated by other means for most important, absolute measurement at this point. Use of the exogenous thermal contrast reagent TmDOTA—has proven less useful thus far. With the exemplary bioheat transfer model, temperature contours in humans can be determined. (See, e.g., References 44 and 51). (See e.g., FIG. 1)

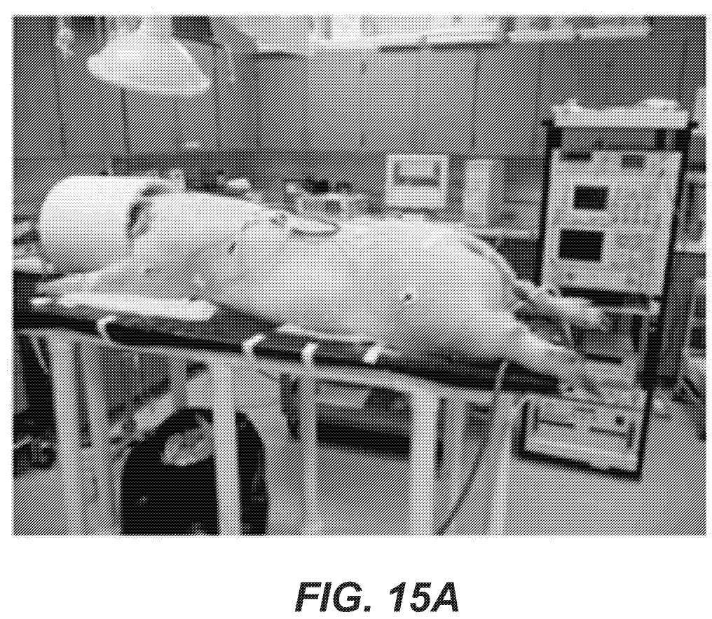

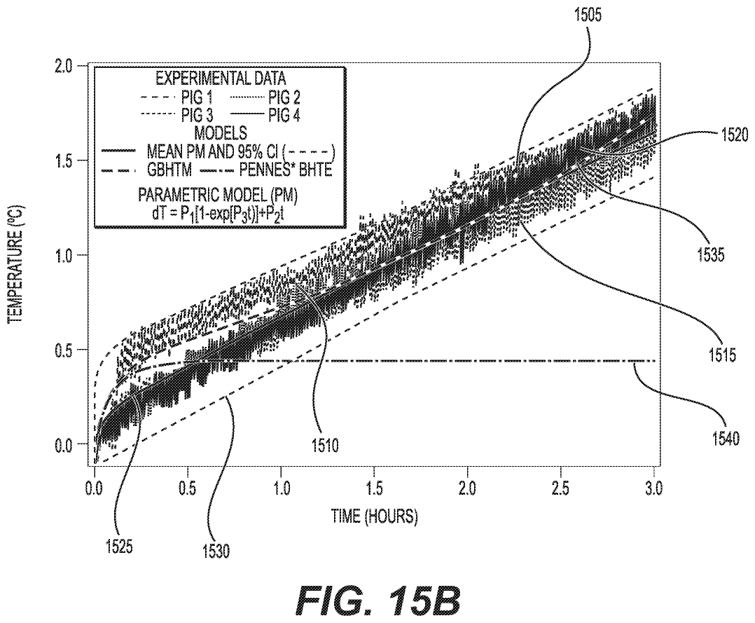

The exemplary bioheat transfer model can provide significant improvements in the exemplary mechanistically derived, thermal model which can account for the heating of blood, vs. the empirically derived Pennes “gold standard” which does not account for blood (e.g., perfusate) heating over time. The exemplary GBHT theoretical model is significantly more accurate and precise than Pennes model, as shown in the exemplary porcine model, providing confidence in the exemplary ability to predict safety in humans. (See e.g., FIGS. 15A and 15B). For example, FIG. 15A illustrates an exemplary image of a pig model. FIG. 15B shows an exemplary graph of the GBHT model according to an exemplary embodiment of the present disclosure, which illustrates a comparison of the exemplary GBHT model vs. the Pennes model for Pig 1 (element 1505), Pig 2 (element 1510), Pig 3 (element 1515), Pig 4 (element 1520), Mean PM (element 1525) and 95% Cl (element 1530), GBHTM (element 1535), and Pennes bioheat thermal equation (element 1540).

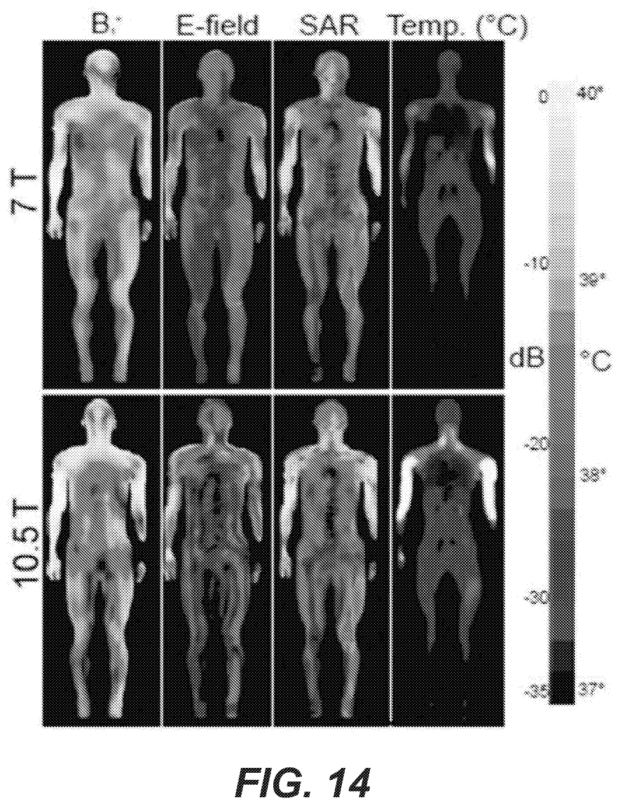

Temperature contours have become increasing non-uniform with higher frequencies. (See e.g., temperature maps shown in FIG. 14). Temperature patterns can be controlled and sometimes mitigates by new multi-channel transmit techniques and beam steering. The exemplary methods and results are already proving useful by being broadly applied for RF safety prediction, assurance and IRB, IDE and manuscript submissions.

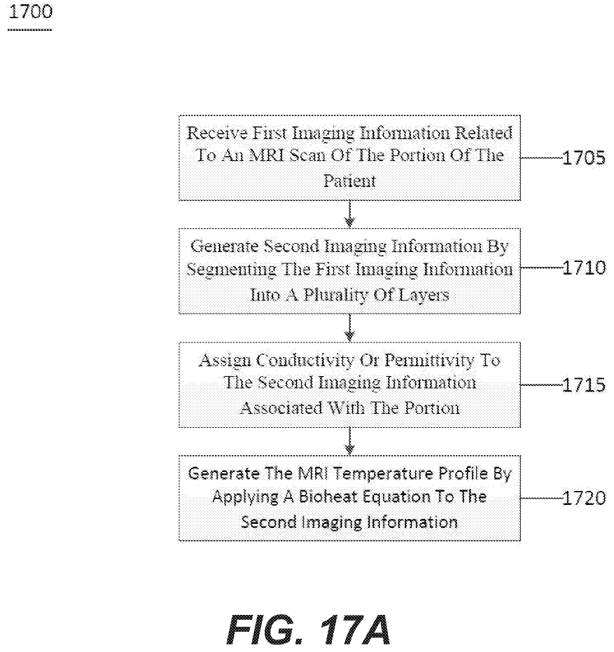

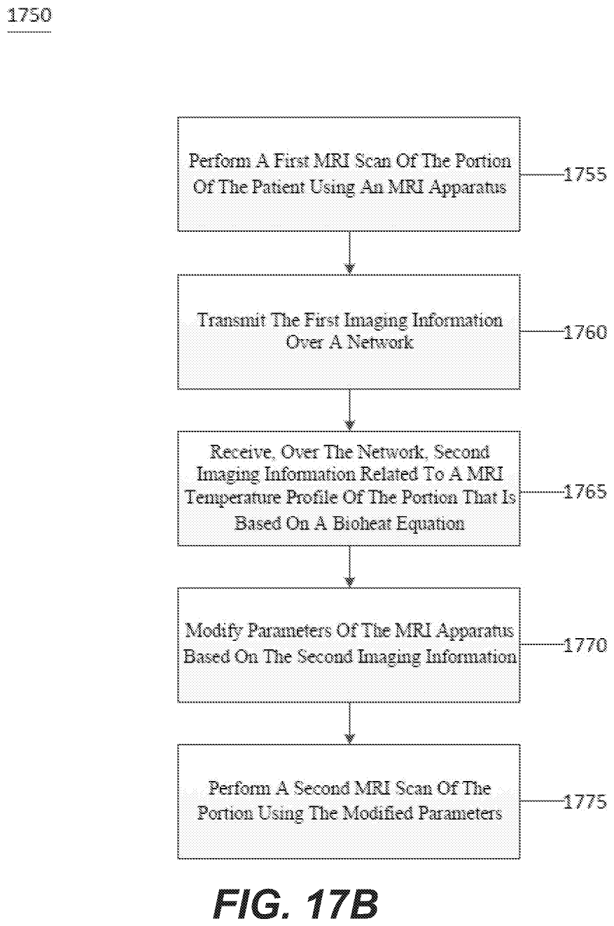

FIG. 17A shows an exemplary flow diagram of an exemplary method 1700 for generating a MRI temperature profile of a portion of a patient according to an exemplary embodiment of the present disclosure. For example, at procedure 1705, first information relating to a MRI scan of the portion of the patient can be received. Second information can be generated at procedure 1710 by segmenting the first information into a plurality of layers. A conductivity or permittivity can be assigned to the second information associated with the portion at procedure 1715. At procedure 1720, the MRI temperature profile can be generated by applying a bioheat equation to the second imaging information.

FIG. 17B illustrates an exemplary flow diagram of the exemplary method 1750 for generating a MRI temperature profile of a portion of a patient according to another exemplary embodiment of the present disclosure. For example, at procedure 1755, a first MRI scan of the portion of the patient can be performed using an MRI apparatus. At procedure 1760, the first imaging information can be transmitted over a network. At procedure 1765, second imaging information can be received over the network that is related to a MRI temperature profile of the portion that is based on a bioheat equation. At procedure 1770, parameters of the MRI apparatus can be modified based on the second imaging information. At procedure 1775, a second MRI scan of the portion can be performed using the modified parameters.

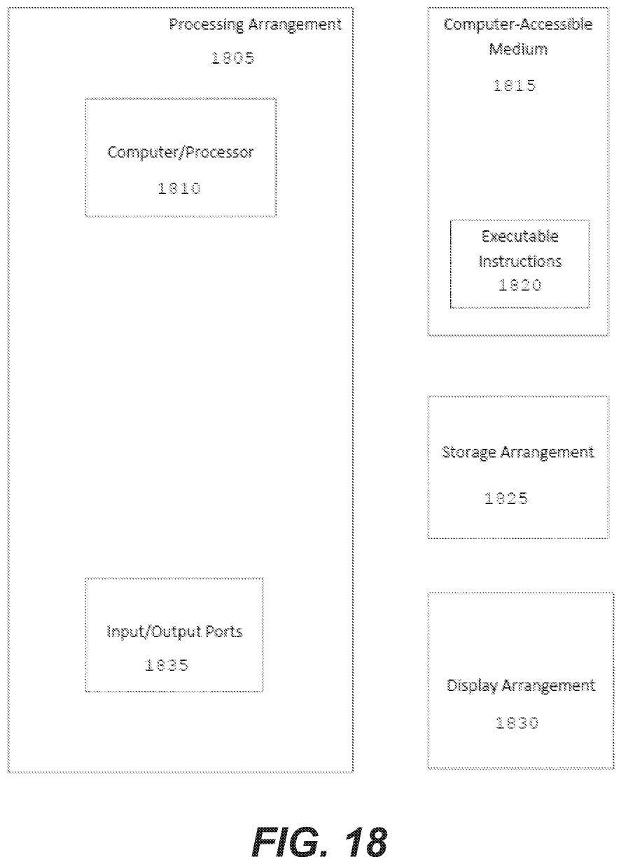

FIG. 18 shows a block diagram of an exemplary embodiment of a system according to the present disclosure. For example, exemplary procedures in accordance with the present disclosure described herein can be performed by a processing arrangement and/or a computing arrangement 1805. Such processing/computing arrangement 1805 can be, for example entirely or a part of, or include, but not limited to, a computer/processor 1810 that can include, for example one or more microprocessors, and use instructions stored on a computer-accessible medium (e.g., RAM, ROM, hard drive, or other storage device).

As shown in FIG. 18, for example a computer-accessible medium 1815 (e.g., as described herein above, a storage device such as a hard disk, floppy disk, memory stick, CD-ROM, RAM, ROM, etc., or a collection thereof) can be provided (e.g., in communication with the processing arrangement 1805). The computer-accessible medium 1815 can contain executable instructions 1820 thereon. In addition or alternatively, a storage arrangement 1825 can be provided separately from the computer-accessible medium 1815, which can provide the instructions to the processing arrangement 1805 so as to configure the processing arrangement to execute certain exemplary procedures, processes and methods, as described herein above, for example.

Further, the exemplary processing arrangement 1805 can be provided with or include an input/output arrangement 1835, which can include, for example a wired network, a wireless network, the internet, an intranet, a data collection probe, a sensor, etc. As shown in FIG. 18, the exemplary processing arrangement 1805 can be in communication with an exemplary display arrangement 1830, which, according to certain exemplary embodiments of the present disclosure, can be a touch-screen configured for inputting information to the processing arrangement in addition to outputting information from the processing arrangement, for example. Further, the exemplary display 1830 and/or a storage arrangement 1825 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

[1] The following references are hereby incorporated by reference in their entireties:
[2] Vaughan J, Nunnally R. A 1.8 T Whole-Body NMR System. 1986 Sep. 24-26; Vista International Hotel, 3 World Trade Center, New York, N.Y. New York Academy of Sciences.
[3] Vaughan J, Hetherington H, Otu J, Pan J, Pohost G. High frequency volume coils for clinical nuclear magnetic resonance imaging and spectroscopy. Magn Reson Med 1994; 32:206-218.
[4] Vaughan J T, Garwood M, Collins C M, Liu W, DelaBarre L, Adriany G, Andersen P, Merkle H, Goebel R, Smith M B, Ugurbil K. 7 T vs. 4 T: RF power, homogeneity, and signal-to-noise comparison in head images. Magn Reson Med 2001; 46(1):24-30.
[5] Vaughan J T, Adriany G, Snyder C J, Tian J, Thiel T, Bolinger L, Liu H, DelaBarre L, Ugurbil K. Efficient high-frequency body coil for high-field MRI. Magn Reson Med 2004; 52(4):851-859.
[6] Vaughan J T, Snyder C J, DelaBarre L J, Bolan P J, Tian J, Bolinger L, Adriany G, Andersen P, Strupp J, Ugurbil K. Whole-body imaging at 7 T: preliminary results. Magn Reson Med 2009; 61(1):244-248.
[7] Vaughan J T, Adriany G, Garwood M, Yacoub E, Duong T, DelaBarre L, Andersen P, Ugurbil K. Detunable transverse electromagnetic (TEM) volume coil for high-field NMR. Magn Reson Med 2002; 47(5):990-1000.
[8] Vaughan T, DelaBarre L, Snyder C, Tian J, Akgun C, Shrivastava D, Liu W, Olson C, Adriany G, Strupp J, Andersen P, Gopinath A, van de Moortele P F, Garwood M, Ugurbil K. 9.4 T human MRI: preliminary results. Magn Reson Med 2006; 56(6):1274-1282.
[9] Vaughan J, Hetherington H, Harrison J, Otu J, Pan J, Noa P, den Hollander J, Pohost G. High frequency coils for clinical nuclear magnetic resonance imaging and spectroscopy. Physica Medica 1993; IX:147-153.
[10] Vaughan J J T; UAB Research Foundation, assignee. Cavity resonator for NMR systems U.S. Pat. No. 5,744,957. 1998 Apr. 28, 1998.
[11] Vaughan J; Massachusetts General Hospital, assignee. RF coil for imaging system. U.S. Pat. No. 6,633,161. 2003.
[12] Vaughan J, Nunnally R. Parallel Arrays for NMR. 1986 Sep. 24-26, 1986; Vista International Hotel, 3 World Trade Center, New York, N.Y. New York Academy of Sciences.
[13] Vaughan J T; Board of Regents, The University of Texas System, assignee. Electrically parallel equal phase resonant loops for nuclear magnetic resonance surface coils U.S. Pat. No. 4,812,761. 1989 Mar. 14, 1989.
[14] Vaughan J, Adriany G, Ugurbil K, Strupp J, Andersen P; Regents of the University of Minnesota, assignee. Parallel Transceiver for Nuclear Magnetic Resonance System. U.S. Pat. No. 6,969,992. 2005.
[15] Vaughan J, Adriany G, Ugurbil K; University of Minnesota, assignee. Shim, gradient, and parallel imaging coil. USA 2003.
[16] Vaughan J T, DelaBarre L, Snyder C, Tian J, Andersen P, Strupp J, Adriany G, van de Moortele P F, Ugurbil K. 9.4 T Human Imaging: Preliminary Results. ISMRM; 2006 May; Seattle. p 529. (ISMRM).
[17] Harrison J, Vaughan J. Finite element modeling of head coils for high frequency magnetic resonance application. 12th Annual Review of Progress in Applied Computational Electromagnetics 1996.
[18] Vaughan J, Harrison J, Hetherington H, Evanochko W, Pohost G. Radiofrequency surface coil heating measurements in porcine muscle. 1992; Berlin. p 4026.
[19] Vaughan J, Harrison J, Thorn B, Pohost G. Hot rings: high frequency heating patterns in tissues. 1993; New York. p 1369.
[20] Vaughan J T. High Frequency Coils. In: Robitaille P, Berliner L, editors. Ultra High Field Magnetic Resonance Imaging. Volume 26. New York: Springer; 2006. p 127-161.
[21] Vaughan J, Lesan B; U Texas Health Sciences Center, assignee. Physiological Standard Phantom for NMR Imaging and Spectroscopy. USA 1989.
[22] Vaughan J T. TEM Body Coils. In: Vaughan J T, Griffiths J, editors. RF Coils for MRI: Wiley-Blackwell; 2012.
[23] Vaughan J, Lemaire C; Live Services LLC and the University of Minnesota, assignee. Active transmit elements for MRI coils and other antenna devices. U.S. Pat. No. 8,604,791. 2013 Dec. 10, 2013.
[24] Snyder C, Vaughan J T, Lemaire C; Life Services LLC, University of Minnesota, assignee. Remotely Adjustable Reactive and resistive Electrical Elements and Methods. USA 2010.
[25] Luo Y, de Graaf R A, DelaBarre L, Tannus A, Garwood M. BISTRO: an outer-volume suppression method that tolerates RF field inhomogeneity. Magnetic Resonance in Medicine. 2001; 45(6):1095-102. PMID: 11378888.
[26] Bolan P J, DelaBarre L, Baker E H, Merkle H, Everson L I, Yee D, Garwood M. Eliminating spurious lipid sidebands in 1H MRS of breast lesions. Magnetic Resonance in Medicine. 2002; 48(2):215-22. PMID: 12210929.
[27] Vaughan J T, Garwood M, Collins C M, Liu W, DelaBarre L, Adriany G, Andersen P, Merkle H, Goebel R, Smith M B, Ugurbil K. 7 T vs. 4 T: RF power, homogeneity, and signal-to-noise comparison in head images. Magnetic Resonance in Medicine. 2001; 46(1):24-30. PMID: 11443707.
[28] Vaughan J T, Adriany G, Snyder C J, Tian J, Thiel T, Bolinger L, Liu H, DelaBarre L, Ugurbil K. Efficient high-frequency body coil for high-field MRI. Magnetic Resonance in Medicine. 2004; 52(4):851-9. PMID: 15389967.
[29] Vaughan T, DelaBarre L, Snyder C, Tian J, Akgun C, Shrivastava D, Liu W, Olson C, Adriany G, Strupp J, Andersen P, Gopinath A, van de Moortele P F, Garwood M, Ugurbil K. 9.4 T human MRI: preliminary results. Magnetic Resonance in Medicine. 2006; 56(6):1274-82. PMID: 17075852.
[30] Vaughan J T, DelaBarre L. TEM Body Coils. Encyclopedia of Magnetic Resonance: John Wiley & Sons, Ltd; 2007.
[31] Akgun C, Delabarre L, Yoo H, Sohn S, Snyder C, Adriany G, Ugurbil K, Gopinath A, Vaughan J. Stepped Impedance Resonators for High Field Magnetic Resonance Imaging. IEEE Transactions on Biomedical Engineering. 2014; 61(2):327-33. PMID: 23508243; PMCID: PMC4142687.
[32] DelaBarre L, Myer D, Vaughan J T. Multi-Channel, In-Bore Power Amplifiers for Multi-Channel Coil at 7 T. Proc Int Soc Reson Med; 2013; Salt Lake City, Utah p 726.
[33] DelaBarre L, Snyder C, Vaughan J, Ugurbil K. A Parallel Transceiver for Human Imaging at 9.4 T. ISMRM. Proceedings 14th Scientific Meeting, International Society for Magnetic Resonance in Medicine; 2006 May; Seattle, Wash. p 130. (ISMRM).
[34] Snyder C, DelaBarre L, Vaughan J T. Automated Tuning and Matching of a 16-Channel TEM Transmit-Only Array used in Conjunction with a 32-Channel Receive-Only Loop Array for MR Cardiac Imaging at 7 Tesla. Microwave Symposium Digest (MTT), 2013 IEEE MTT-S International; 2013 Jun. 2-7, 2013; Seattle, Wash. p 1-4.
[35] Sohn S-M, DelaBarre L, Gopinath A, Vaughan J T. Automatically Tuned and Matched RF Transceive Head Coil at 7 T. Proceedings 22nd Scientific Meeting, International Society for Magnetic Resonance in Medicine; 2014; Milan, IT. p 318.
[36] Snyder C J, DelaBarre L, Metzger G J, van de Moortele P F, Akgun C, Ugurbil K, Vaughan J T. Initial results of cardiac imaging at 7 Tesla. Magnetic Resonance in Medicine. 2009; 61(3):517-24. PMID: 19097233; PMCID: PMC2939145.
[37] DelaBarre L, Snyder C, Van De Moortele P-F, Akgun C, Ugurbil K, Vaughan J T. Cardiac Imaging at 7 T. ISMRM. Proceedings 15th Scientific Meeting, International Society for Magnetic Resonance in Medicine; 2007 May 19-25, 2007; Berlin, Del. p 3867. (ISMRM).
[38] Snyder C, DelaBarre L, Metzger G, Ugurbil K, Vaughan J T. 32-Channel Receive Only Array for Cardiac Imaging at 7 T. Proceedings 19th Scientific Meeting, ISMRM; 2011; Montreal, Quebec. p 165.
[39] DelaBarre L J, Snyder C J, Vaughan J T, van de Moortele P F. Balanced SSFP cardiac imaging at 7 T. Proceedings 19th Scientific Meeting, ISMRM; 2011; Montreal, Quebec. p 596.
[40] Schmitter S, DelaBarre L, Wu X, Greiser A, Wang D, Auerbach E J, Vaughan J T, Ugurbil K, Van de Moortele P-F. Cardiac imaging at 7 tesla: Single- and two-spoke radiofrequency pulse design with 16-channel parallel excitation. Magnetic Resonance in Medicine. 2013; 70(5):1210-9. PMID: 24038314; PMCID: PMC3960017.
[41] Akgun C, DelaBarre L, Yoo H, Snyder C, Gopinath A, Ugurbil K, Vaughan J T. Stepped Impedance Resonators for High Field MRI. Proceedings 19th Scientific Meeting, ISMRM; 2011; Montreal, Quebec. p 3815.
[42] Akgun C, Yoo H, DelaBarre L, Snyder C, Adriany G, Van de Moortele P F, Gopinath A, Ugurbil K, Vaughan J T. Novel 24 Element Multi-Transmit Volume Coil for High Field MRI. Proceedings 19th Scientific Meeting, ISMRM; 2011; Montreal, Quebec. p 3814.
[43] Crandall C G, Brothers R M, Zhang R, Brengelmann G L, Covaciu L, Jay O, Cramer M N, Fuller A, Maloney S K, Mitchell D, Romanovsky A A, Caputa M, Nordstrom C H, Reinstrup P, Nishiyasu T, Fujii N, Hayashi K, Tsuji B, Flouris A D, Cheung S S, Vagula M C, Nelatury C F, Choi J H, Shrivastava D, Gordon C J, Vaughan J T. Comments on point:counterpoint: humans do/do not demonstrate selective brain cooling during hyperthermia. J Appl Physiol (1985). 2011; 110(2):575-80. PMID: 21304015.
[44] DelaBarre L, Snyder C, Van de Moortele P F, Vaughan J T. Cardiac Functional Imaging at the CMRR's 7 T. The 3rd Siemens Ultra High Field User Meeting; 2011 Oct. 17, 2011; Minneapolis, Minn.
[45] DelaBarre L J, Snyder C J, Vaughan J T, van de Moortele P F. Balanced SSFP cardiac imaging at 7 T. Proceedings 19th Scientific Meeting, ISMRM; 2011; Montreal, Quebec. p 596.
[46] Metzger G, DelaBarre L, Bi X, Shah S, Zuehlsdorff S, Vaughan J T, Ugurbil K, Van de Moortele P F. Left Coronary Artery Imaging at 7 T: Initial Results using Multiple B1+ Shimming Algorithms and Targets. Proceedings 19th Scientific Meeting, ISMRM; 2011; Montreal, Quebec. p 116.
[47] Rodgers C, Piechnik S, DelaBarre L, Van de Moortele P F, Snyder C, Neubauer S, Robson M, Vaughan J T. Human cardiac T1 measured at 7 Tesla. Proceedings 19th Scientific Meeting, ISMRM; 2011; Montreal, Quebec. p 615.
[48] Shrivastava D, Goerke U, Michaeli S, Tian J, Abosch A, Vaughan J T. An MR Thermometry-GBHTM ‘Hybrid’ Model to Determine Radiofrequency Heating near Implanted Leads in High Field Imaging. Proceedings 19th Scientific Meeting, ISMRM; 2011; Montreal, Quebec. p 3767.
[49] Shrivastava D, Goerke U, Michaeli S, Tian J, DelaBarre L, Vaughan J T. Total Proton Resonance Frequency Shift Coefficient in the Porcine Brain to Image Radiofrequency Heating in Ultra-high Field MRI. Proceedings 19th Scientific Meeting, ISMRM; 2011; Montreal, Quebec. p 497.
[50] Shrivastava D, Gordon C J, Vaughan J T. Selective Brain Cooling in Humans during Exercise. Journal of Applied Physiology. 2011; 110(2):580-. PMID: ISI:000288207100049.
[51] Shrivastava D, Hanson T, Goerke U, Abosch A, Vaughan J T. Radiofrequency Heating near Deep Brain Stimulation Lead Electrodes during MRI at 3 T. FDA Magnetic Resonance Imaging (MRI) Safety Public Workshop; 2011; Silver Spring, Md., USA.
[52] Shrivastava D, Hanson T, Kulesa J, Tian J, Adriany G, Vaughan J T. Radiofrequency heating in porcine models with a “large” 32 cm internal diameter, 7 T (296 MHz) head coil. Magnetic Resonance in Medicine. 2011; 66(1):255-63. PMID: 21337423; PMCID: PMC3339408.
[53] Shrivastava D, Kulesa J, Hanson T, Tian J, DelaBarre L, Vaughan J T. In Vivo Radiofrequency Heating during Magnetic Resonance Imaging at High and Ultra-High Fields. FDA Magnetic Resonance Imaging (MRI) Safety Public Workshop; 2011 Oct. 25-26, 2011; Silver Spring, Md., USA.
[54] Shrivastava D, Kulesa J, Hanson T, Tian J, Vaughan J T. Radio-frequency heating due to a 9′ ID, 8 Channel 7 T (296 MHz) Head Coil. The 8th Biennial Minnesota Workshops 2011; 2011; Minneapolis, Minn., USA.
[55] Shrivastava D, Kulesa J, Tian J, Adriany G, DelaBarre L, Vaughan J T. Radio-Frequency Heating in Swine with an 8 Channel, 7 T (296 MHz) Head Coil. Proceedings 19th Scientific Meeting, ISMRM; 2011; Montreal, Quebec. p 3822.
[56] Shrivastava D, Tian J, Abosch A, Vaughan J T. Radio-Frequency Heating at Deep Brain Stimulation Lead Electrodes due to Imaging with Head Coils in 3 T and 7 T. Proceedings 19th Scientific Meeting, ISMRM; 2011; Montreal, Quebec. p 3764.
[57] Shrivastava D, Vaughan J T. RF Heating In Vivo in High Field MRI. ISMRM Workshop: Ultra-high field systems and applications 7 T and beyond; 2011; Lake Louis, Alberta, Calif.
[58] Snyder C, DelaBarre L, Metzger G, Ugurbil K, Vaughan J T. 32-Channel Receive Only Array for Cardiac Imaging at 7 T. Proceedings 19th Scientific Meeting, ISMRM; 2011; Montreal, Quebec. p 165.
[59] Snyder C, Rodgers C, DelaBarre L, Robson M, Vaughan J T. Remotely Tuned and Matched Eight-Channel Transceive Head Array Using Piezoelectric Actuators. The 8th Biennial Minnesota Workshops 2011; 2011 Oct. 14-16, 2011; Minneapolis, Minn., USA.
[60] Snyder C, Rogers C, DelaBarre L, Robson M, Vaughan J T. Remote Tuning and Matching an 8-Channel Transceive Array at 7 T. Proceedings of the 19th Annual Meeting of ISMRM; 2011; Montreal, Quebec.
[61] Sohn S M, Vaughan J T, Gopinath A. An Interdigitated Split-Ring Resonator for Metamaterials. Microwave and Optical Technology Letters. 2011; 53(1):174-7. PMID: ISI:000285231200048.
[62] Sohn S-M, Gopinath A, Vaughan J T. Electrically auto-tuned RF coil design. Proceedings 19th Scientific Meeting, International Society for Magnetic Resonance in Medicine; 2011; Montreal, Quebec.
[63] Sung-Min S, Vaughan J T, Gopinath A. Auto-tuning of the RF transmission line coil for high-fields magnetic resonance imaging (MRI) systems. Microwave Symposium Digest (MTT), 2011 IEEE MTT-S International; 2011 5-10 Jun. 2011. p 1-4.
[64] Suttie J J, DelaBarre L, Pitcher A, Van de Moortele P F, Dass S, Snyder C, Francis J M, Metzger G J, Weale P, Ugurbil K, Neubauer S, Robson M D, Vaughan J T. Ultrahigh field (7 Tesla) human cardiovascular magnetic resonance imaging to assess cardiac volumes and mass: A feasibility and validation study. In Proceedings Society of Cardiovascular Magnetic Resonance. 2011.
[65] Tian J, Gopinath A, Vaughan J T. Tailoring RF Power Distribution for Body Torso MRI at 300 MHz. Proceedings 19th Scientific Meeting, ISMRM; 2011; Montreal, Quebec. p 3851.
[66] Vaughan J T, Adriany G, Ugurbil K, inventors; Regents of the University of Minnesota, assignee. Assymetric radio frequency magnetic line array 2011 Feb. 22, 2011.
[67] Vaughan J T, Myer D. RF Coil Element Mounted Power Amplifiers. Proceedings 19th Scientific Meeting, International Society for Magnetic Resonance in Medicine; 2011; Montreal, Quebec. p 1851.
[68] Wu X, Schmitter S, Tian J, Vaughan J T, Ugurbil K, Van de Moortele P F. SAR Analysis of Parallel Transmission in Cardiac Imaging at 7 T. Proceedings 19th Scientific Meeting, ISMRM; 2011; Montreal, Quebec. p 492.
[69] Yoo H, Vaughan J T, Gopinath A. RF (B1) Field Calculation with Transmission Line Resonator Analysis for HIgh-Field Magnetic Resonance Systems. IEEE Antennas and Wireless Propagation Letters. 2011; 10(3).
[70] Adriany G, Waks M, Tramm B, Schillak S, Yacoub E, de Martino F, Van de Moortele P-F, Nasclaris T, Olman C, Vaughan J T, Ugurbil K. An Open Faced 4 ch. Loop Transmit/16ch. Receive Array Coil for HiRes fMRI at 7 Tesla. Proc Int Soc Reson Med; 2012; Melbourne, AU. p 429.
[71] Akgun C, DelaBarre L, Snyder C, Ugurbil K, Gopinath A, Vaughan J T. Optimizing TEM Transceiver Elements at 7 Tesla. Proc Int Soc Reson Med; 2012; Melbourne, AU. p 2781.
[72] Akgun C E, Snyder C, DelaBarre L, Moeller S, Van de Moortele P-F, Vaughan J T, Ugurbil K, inventors; Regents of the University of Minnesota, assignee. Three dimensional RF coil structures for field profiling U.S. Pat. No. 8,193,809. 2012 Jun. 5, 2012.
[73] DelaBarre L, Van de Moortele P F, Snyder C J, Tian J, Moeller S, Vaughan J T. Methodology for UHF multichannel coil evaluation. Proc Int Soc Reson Med; 2012; Melbourne, AU. p 2616
[74] Ellermann J, Goerke U, Morgan P, Ugurbil K, Tian J, Schmitter S, Vaughan T, Van De Moortele P F. Simultaneous bilateral hip joint imaging at 7 Tesla using fast transmit B(1) shimming methods and multichannel transmission—a feasibility study. NMR in Biomedicine. 2012 Epub before Print DOI: 10.1002/nbm.2779. PMID: 22311346.
[75] Gopinath A, Ebbini E, Vaughan J T. Digital Beam Forming In MRI. Proc Int Soc Reson Med; 2012; Melbourne, AU. p 2678.
[76] Olson C, Yoo H, Delabarre L, Vaughan J T, Gopinath A. RF B1 field localization through convex optimization. Microwave and Optical Technology Letters. 2012; 54(1):31-7.
[77] Rodgers C T, Piechnik S K, DelaBarre L, Van de Moortele P-F, Snyder C J, Neubauer S, Robson M D, Vaughan J T. Human cardiac T1 mapping in vivo at 7 T: quantifying and correcting for partial inversion. Proc Int Soc Reson Med; 2012; Melbourne, AU. p 3780.
[78] Schillak S M, Vaughan J T, Lemaire C A, Waks M T, inventors; Schillak Scott M, Vaughan Jr John Thomas, Lemaire Charles A, Waks Matthew T, assignee. Simultaneous Tx-Rx for MRI Systems and Other Antenna Devices. USA 2012.
[79] Shrivastava D, Abosch A, Hughes J, Goerke U, DelaBarre L, Visaria R, Harel N, Vaughan J T. Heating induced near deep brain stimulation lead electrodes during magnetic resonance imaging with a 3 T transceive volume head coil. Physics in Medicine and Biology. 2012; 57(17):5651-65. PMID: 22892760; PMCID: PMC3469254.
[80] Shrivastava D, Hanson T, Goerke U, DelaBarre L, Visaria R, Iaizzo P, Abosch A, Vaughan J T. Heating near Deep Brain Stimulation (DBS) Lead Electrodes during imaging with a 3 T Transceive Head Coil in Cadaveric Porcine Heads. Proc Int Soc Reson Med; 2012; Melbourne, AU. p 2730.
[81] Shrivastava D, Utecht L, Tian J, Hanson T, Vaughan J T. Radiofrequency Heating in Swine due to a 3 T (123.2 MHz) and 7 T (296 MHz) Head Coil. Proc Int Soc Reson Med; 2012; Melbourne, AU. p 2670.
[82] Shrivastava D, Vaughan J T. Radiofrequency Heating Models and Measurements. In: Vaughan J T, Griffiths J, editors. RF Coils for MRI: Wiley-Blackwell; 2012.
[83] Snyder C J, Delabarre L, Moeller S, Tian J, Akgun C, Van de Moortele P F, Bolan P J, Ugurbil K, Vaughan J T, Metzger G J. Comparison between eight- and sixteen-channel TEM transceive arrays for body imaging at 7 T. Magnetic Resonance in Medicine. 2012; 67(4):954-64. PMID: 22102483; PMCID: PMC3290686.
[84] Snyder C J, Vaughan J T, Lemaire C A, inventors; Life Services, LLC, assignee. Remotely Adjustable Reactive and Resistive Electrical Elements and Method. U.S. Pat. No. 8,299,681. 2012 Oct. 30, 2012
[85] Sohn S-M, DelaBarre L, Vaughan J T, Gopinath A. P (Pi)-matching Technique for RF Coil of MRI Systems. IEEE Microwave Theory and Techniques International Microwave Symposium; 2012; Montréal, CA.
[86] Sung-Min S, DelaBarre L, Vaughan J T, Gopinath A. Π (Pi)-matching technique for RF coil of MRI systems. Microwave Symposium Digest (MTT), 2012 IEEE MTT-S International; 2012 17-22 Jun. 2012. p 1-3.
[87] Sung-Min S, DelaBarre L, Vaughan J T, Gopinath A. RF multi-channel head coil design with improved B1+ Fields uniformity for high field MRI systems. Microwave Symposium Digest (MTT), 2012 IEEE MTT-S International; 2012 17-22 Jun. 2012. p 1-3.
[88] Suttie J J, Delabarre L, Pitcher A, van de Moortele P F, Dass S, Snyder C J, Francis J M, Metzger G J, Weale P, Ugurbil K, Neubauer S, Robson M, Vaughan T. 7 Tesla (T) human cardiovascular magnetic resonance imaging using FLASH and SSFP to assess cardiac function: validation against 1.5 T and 3 T. NMR in Biomedicine. 2012; 25(1):27-34. PMID: 21774009; PMCID: PMC3440016.
[89] Tian J, Shrivastava D, Strupp J, Zhang J, Vaughan J T. From 7 T to 10.5 T: B1+, SAR and Temperature Distribution for Head and Body MRI. Proc Int Soc Reson Med; 2012; Melbourne, AU. p 2666.
[90] Vaughan J T, inventor; Regents of the University of Minnesota, assignee. Multi-channel RF coil system with multi-channel RF coil transceiver detecting more than one frequency at the same time for magnetic resonance imaging systems and methods. U.S. Pat. No. 8,217,653. 2012 Jul. 10, 2012.
[91] Vaughan J T, DelaBarre L. TEM Body Coils. In: Vaughan J T, Griffiths J, editors. RF Coils for MRI: Wiley-Blackwell; 2012.
[92] Vaughan J T, Griffiths JR, editors. RF Coils for MRI: John Wiley and Sons Publishing; 2012.
[93] Vaughan J T, Lemaire C A, inventors; Life Services, LLC, Regents Of The University Of Minnesota, assignee. Method and Coils for Human Whole-Body Imaging at 7 T 2012 Aug. 5, 2011 (Filed).
[94] Yoo H, Gopinath A, Vaughan J T. A method to localize RF B(1) field in high-field magnetic resonance imaging systems. IEEE Transactions on Biomedical Engineering. 2012; 59(12):3365-71. PMID: 22929360; NIHMSID: 580388.
[95] Adriany G, Schillak S, Waks M, Tramm B, Roebroeck A, Formisano E, DeMartino F, Yacoub E S, Vaughan J T. A Flexible 4 Ch. Transmit/16ch. Receive Auditory Cortex Array for HiRes fMRI at 7 Tesla. Proc Int Soc Reson Med; 2013; Salt Lake City, Utah p 2734.
[96] DelaBarre L, Myer D, Vaughan J T. Multi-Channel, In-Bore Power Amplifiers for Multi-Channel Coil at 7 T. Proc Int Soc Reson Med; 2013; Salt Lake City, Utah p 726.
[97] Hess A T, Snyder C J, Keith G A, Rodgers C T, Neubauer S, Vaughan J T, Robson M D. Coil Tuning with Piezoelectric Actuators Using the MRI Signal as the Optimization Parameter. Proc Int Soc Reson Med; 2013; Salt Lake City, Utah p 2745.
[98] Keith G A, Rodgers C T, Hess A T, Snyder C J, Vaughan J T, Robson M D. Computerised Tuning of an 8-Channel Cardiac TEM Array at 7 T: An Integrated System Using Piezoelectric Actuators and Power Monitors. Proc Int Soc Reson Med; 2013; Salt Lake City, Utah p 2743.
[99] Rodgers C T, Clarke W T, Snyder C, Vaughan J T, Neubauer S, Robson M D. Human Cardiac 31P Magnetic Resonance Spectroscopy at 7 Tesla. Proc Int Soc Reson Med; 2013; Salt Lake City, Utah p 578.
[100] Rodgers C T, Keith G A, Hess A T, Snyder C, Vaughan J T, Robson M D. An Algorithm for Automatic Optimisation of Transmit Array Coil Tune and Match Applied in a Cardiac TEM Coil at 7 T. Proc Int Soc Reson Med; 2013; Salt Lake City, Utah p 4402.
[101] Rodgers C T, Piechnik S K, Delabarre L J, Van de Moortele P F, Snyder C J, Neubauer S, Robson M D, Vaughan J T. Inversion recovery at 7 T in the human myocardium: measurement of T1, inversion efficiency and B1. Magnetic Resonance in Medicine. 2013; 70(4):1038-46. PMID: 23197329; PMCID: PMC4134266.
[102] Schmitter S, DelaBarre L, Wu X, Greiser A, Wang D, Auerbach E J, Vaughan J T, Ugurbil K, Van de Moortele P-F. Cardiac imaging at 7 tesla: Single- and two-spoke radiofrequency pulse design with 16-channel parallel excitation. Magnetic Resonance in Medicine. 2013; 70(5):1210-9. PMID: 24038314; PMCID: PMC3960017.
[103] Shrivastava D, Tian J, Hughes J, Vaughan J T. Stepping towards Subject-Specific Temperature Modeling to Improve Thermal Safety in Clinical and Ultra-high Field MRI. Proceedings of the ASME/FDA 2013 1st Annual Frontiers in Medical Devices: Applications of Computer Modeling and Simulations; 2013 Sep. 10-12, 2013; Washington D.C., USA. p V001 T08A2.
[104] Shrivastava D, Utecht L, Tian J, Visaria R, Hughes J, Vaughan J T. In vivo Radio-Frequency Heating Did Not Change Due to the Power Deposition from Similar 3 T and 7 T Head Coils. Proc Int Soc Reson Med; 2013; Salt Lake City, Utah p 284.
[105] Shrivastava D, Utecht L, Tian J, Visaria R, Hughes J, Vaughan J T. Radiofrequency Heating During Head Imaging in a 3 T Transmit Body Coil. Proc Int Soc Reson Med; 2013; Salt Lake City, Utah p 2828.
[106] Snyder C, DelaBarre L, Hess A T, Rodgers C, Robson M, Vaughan J T. A Separated Transmit-Only, Receive-Only Array for Body Imaging at 7 T with Automated Tuning and Matching Capabilities. Proc Int Soc Reson Med; 2013; Salt Lake City, Utah p 131.
[107] Snyder C, DelaBarre L, Vaughan J T. Automated Tuning and Matching of a 16-Channel TEM Transmit-Only Array used in Conjunction with a 32-Channel Receive-Only Loop Array for MR Cardiac Imaging at 7 Tesla. Microwave Symposium Digest (MTT), 2013 IEEE MTT-S International; 2013 Jun. 2-7, 2013; Seattle, Wash. p 1-4.
[108] Snyder C J, Vaughan J T, Lemaire C A, inventors; Regents Of The University Of Minnesota, Life Services, LLC, assignee. Method and Remotely Adjustable Reactive and Resistive Electrical Elements 2013 Oct. 30, 2012.
[109] Sohn S, Vaughan J T, Gopinath A, inventors; System and Method for Control of RF Circuits for Use With an MRI System 2013.
[110] Sohn S-M, DelaBarre L, Gopinath A, Vaughan J T. RF Coil Design with Automatic Tuning and Matching. Proc Int Soc Reson Med; 2013; Salt Lake City, Utah p 731.
[111] Sohn S-M, DelaBarre L, Vaughan J T, Gopinath A. 8-Channel RF Head Coil of MRI with Automatic Tuning and Matching Microwave Symposium Digest (MTT), 2013 IEEE MTT-S International; 2013 Jun. 2-7, 2013; Seattle, Wash. p 1-4.
[112] Son H W, Cho Y K, Gopinath A, Vaughan J T, Lee C H, Yoo H. B1 shimming with SAR reduction in high-field MRI. Journal of Electromagnetic Waves and Applications. 2013; 27(12):1521-4. NIHMSID: 580390.
[113] Tian J, Shrivastava D, Adriany G, Strupp J, Schillak S, Zhang J, Ugurbil K, Vaughan JT. Comparison of 3 RF Head Arrays for 7 T MRI. Proc Int Soc Reson Med; 2013; Salt Lake City, Utah p 2758.
[114] Vaughan J T, Adriany G, Snyder C, Tian J, Ugurbil K, van de Moortele P-F, Moeller S, inventors; Regents of the University of Minnesota, assignee. Multi-Current Elements for Magnetic Resonance Radio Frequency Coils. Canada 2013.
[115] Vaughan J T, Adriany G, Snyder C, Tian J, Ugurbil K, van de Moortele P-F, Moeller S, inventors; Regents of the University of Minnesota, assignee. Multi-Current Elements for Magnetic Resonance Radio Frequency Coils. Japan 2013.
[116] Vaughan J T, DelaBarre L, Tian J, Sohn S, Shrivastava D, Adriany G, Ugurbil K. RF technology for human MRI at 10.5 T. Microwave Workshop Series on RF and Wireless Technologies for Biomedical and Healthcare Applications (IMWS-BIO), 2013 IEEE MTT-S International; 2013 9-11 Dec. 2013. p 1-3.
[117] Vaughan J T, Lemaire C A, inventors; Life Services, LLC; Regents of the University of Minnesota, assignee. Active transmit elements for MRI coils and other antenna devices. U.S. Pat. No. 8,604,791. 2013 Dec. 10, 2013.
[118] Vaughan J T, Tian J, inventors; University of Minnesota, assignee. Coil Element Decoupling for MRI. U.S. Pat. No. 8,380,266. 2013 Feb. 19, 2013.
[119] Wu X, Tian J, Schmitter S, Hanna B, Pfeuffer J, Hamm M, Nistler J, Vaughan J T, Ugurbil K, Van de Moortele P-F. Z-Shim RF Coil Design Enhances Parallel Transmit Performance in Body Imaging at 3 T. Proc Int Soc Reson Med; 2013; Salt Lake City, Utah p 4398.
[120] Yoo H, Lee J, Akgun C E, Gopinath A, Vaughan J T. B1 field comparison for RF coils in ultra-high-field MRI. Electronics Letters. 2013; 49(25):1601-3. NIHMSID: 580389.
[121] Adriany G, Schillak S, Waks M, Tramm B, Vaughan T, Ugurbil K, Van de Moortele P-F, Schmitter S. A 16-Channel Arterial Spin Labeling—Head Transceiver Array Combination for 7 Tesla. Proceedings 22nd Scientific Meeting, International Society for Magnetic Resonance in Medicine; 2014; Milan, IT. p 317.
[122] Akgun C, Delabarre L, Yoo H, Sohn S, Snyder C, Adriany G, Ugurbil K, Gopinath A, Vaughan J. Stepped Impedance Resonators for High Field Magnetic Resonance Imaging. IEEE Transactions on Biomedical Engineering. 2014; 61(2):327-33. PMID: 23508243; PMCID: PMC4142687.
[123] Corum C A, Benson J C, Idiyatullin D, Snyder A L, Snyder C J, Hutter D, Everson L I, Eberly L E, Nelson M T, Garwood M. High-Spatial- and High-Temporal-Resolution Dynamic Contrast-enhanced MR Breast Imaging with Sweep Imaging with Fourier Transformation: A Pilot Study. Radiology. 2014 Epub before Print DOI: 10.1148/radiol.14131273:131273. PMID: 25247405.
[124] Corum C A, Idiyatullin D, Snyder C J, Garwood M. Gap cycling for SWIFT. Magnetic Resonance in Medicine. 2014 Epub before Print DOI: 10.1002/mrm.25141. PMID: 24604286; PMCID: PMC4143500.
[125] Eryaman Y, Guerin B, Akgun C, Herraiz J L, Martin A, Torrado-Carvajal A, Malpica N, Hernandez-Tamames J A, Schiavi E, Adalsteinsson E, Wald L L. Parallel transmit pulse design for patients with deep brain stimulation implants. Magnetic Resonance in Medicine. 2014 Epub before Print DOI: 10.1002/mrm.25324. PMID: 24947104.
[126] Keith G A, Rodgers C T, Hess A, Snyder C J, Vaughan J T, Robson M. A Fully Integrated Automatic Tune and Match System for an 8-Channel Transmit/Receive Cardiac TEM Array at 7 T: Initial Results in a Phantom and Volunteers. Proceedings 22nd Scientific Meeting, International Society for Magnetic Resonance in Medicine; 2014; Milan, IT. p 1339.
[127] Keith G A, Rodgers C T, Hess A T, Snyder C J, Vaughan J T, Robson M D. Automated tuning of an eight-channel cardiac transceive array at 7 Tesla using piezoelectric actuators. Magnetic Resonance in Medicine. 2014 Epub before Print DOI: 10.1002/mrm.25356. PMID: 24986525; PMCID: PMC4245186.
[128] Metzger G, Elliott I, Tian J, Shrivastava D, Van de Moortele P-F, Adriany G. Development and Evaluation of a Solid Endorectal Coil for 7 Tesla. Proceedings 22nd Scientific Meeting, International Society for Magnetic Resonance in Medicine; 2014; Milan, IT. p 626.
[129] Rodgers C T, Clarke W T, Snyder C, Vaughan J T, Neubauer S, Robson M D. Human cardiac 31P magnetic resonance spectroscopy at 7 tesla. Magnetic Resonance in Medicine. 2014; 72(2):304-15. PMID: 24006267; PMCID: PMC4106879.
[130] Schmitter S, Wu X, Ugurbil K, Van de Moortele P F. Design of parallel transmission radiofrequency pulses robust against respiration in cardiac MRI at 7 Tesla. Magnetic Resonance in Medicine. 2014 Epub before Print DOI: 10.1002/mrm.25512. PMID: 25411131; PMCID: PMC4437923.
[131] Shrivastava D, Tian J, Hughes J, Vaughan J T. Radiofrequency Heating During Body Imaging in a 3 T Body Coil and Patient Safety. Proceedings 22nd Scientific Meeting, International Society for Magnetic Resonance in Medicine; 2014; Milan, IT. p 172.
[132] Shrivastava D, Utecht L, Tian J, Hughes J, Vaughan J T. In vivo radiofrequency heating in swine in a 3 T (123.2-MHz) birdcage whole body coil. Magnetic Resonance in Medicine. 2014; 72(4):1141-50. PMID: 24259413; PMCID: PMC4041852.
[133] Shrivastava D, Vaughan J T, Visaria R. Underestimation Error in Estimating ‘True’ Local SAR in Perfused Tissues in High and Ultra-High Field MRI. Proceedings 22nd Scientific Meeting, International Society for Magnetic Resonance in Medicine; 2014; Milan, IT. p 322.
[134] Snyder A L S, Snyder C J, Garwood M G, Vaughan J T J, inventors; Beam steering with resonance along a trajectory. USA 2014 Aug. 7, 2014.
[135] Sohn S M, DelaBarre L, Gopinath A, Vaughan J T. Design of an Electrically Automated RF Transceiver Head Coil in MRI. IEEE Trans Biomed Circuits Syst. 2014 Epub before Print DOI: 10.1109/TBCAS.2014.2360383. PMID: 25361512; PMCID: PMC4412778.
[136] Sohn S-M, DelaBarre L, Gopinath A, Vaughan J T. Automatically Tuned and Matched RF Transceive Head Coil at 7 T. Proceedings 22nd Scientific Meeting, International Society for Magnetic Resonance in Medicine; 2014; Milan, IT. p 318.
[137] Sohn S-m, DelaBarre L, Gopinath A, Vaughan J T. RF Head Coil Design With Improved RF Magnetic Near-Fields Uniformity for Magnetic Resonance Imaging (MRI) Systems. Microwave Theory and Techniques, IEEE Transactions on. 2014; 62(8):1784-9. PMID: 25892746; PMCID: PMC4399018.
[138] Sohn S-M, Gopinath A, Vaughan J T. Tunable and High Directivity Coupler for MRI Applications. Microwave Symposium Digest (MTT), 2014 IEEE MTT-S International; 2014; Tampa, Fla. IEEE.
[139] Tian J, DelaBarre L, Vaughan J T. Coil Evaluation Metrics. Proceedings 22nd Scientific Meeting, International Society for Magnetic Resonance in Medicine; 2014; Milan, IT. p 1381.
[140] Vaughan J T. RF (B1 & E) Fields for MRI: Overview. Safety in MRI: Guidelines, Rationale & Challenges; 2014 Sep. 4-7, 2014; Washington, D.C.
[141] Vaughan J T, DelaBarre L, Tian J, Sohn S, Shrivastava D, Adriany G, Ugurbil K. Toward 10.5 T MRI. Proceedings 22nd Scientific Meeting, International Society for Magnetic Resonance in Medicine; 2014; Milan, IT. p 4822.
[142] Vaughan J T, Tian J. Improved RF Transmit Coil and Methods for MRI. Interventional MRI Symposium; 2014 Oct. 10-11, 2014; Leipzig, Del.
[143] Vaughan J T, Tian J, Shrivastava D. Understanding RF Safety for MRI. Interventional MRI Symposium; 2014 Oct. 10-11, 2014; Leipzig, Del.
[144] Wu X, Tian J, Schmitter S, Vaughan T, Ugurbil K, Van de Moortele P-F. Z-Stacked RF Array Design Enhances Parallel Transmit Multiband RF Performance in Whole Brain Simultaneous Multislice Imaging at 7 T. Proceedings 22nd Scientific Meeting, International Society for Magnetic Resonance in Medicine; 2014; Milan, IT. p 543.
[145] Zhang X, Wu X, Tian J, Schmitter S, DelaBarre L, Hamm M, Pfeuffer J, Wang D, He B, Vaughan T, Ugurbil K, Van de Moortele P-F. Excitation Performance and SAR Control with Z-Stacked Body RF Coil Arrays in Parallel Transmission at 3 T. Proceedings 22nd Scientific Meeting, International Society for Magnetic Resonance in Medicine; 2014; Milan, IT. p 4882.
[146] Adriany G, Schillak S, Waks M, Tramm B, Grant A, Yacoub E, Vaughan J T, Olman C, Schmitter S, Ugurbil K. A Modular 16 Ch. Transmit/32 Ch. Receive Array for Parallel Transmission and High Resolution fMRI at 7 Tesla. Proceedings 23rd Scientific Meeting, International Society for Magnetic Resonance in Medicine; 2015; Toronto, ON. p 622.
[147] Boulant N, Wu X, Adriany G, Schmitter S, Ugurbil K, Van de Moortele P-F. Direct Control of the Temperature Rise in Parallel Transmission Via Temperature Virtual Observation Points: Simulations at 10.5 T. Proceedings 23rd Scientific Meeting, International Society for Magnetic Resonance in Medicine; 2015; Toronto, ON. p 548.

1. A non-transitory computer-accessible medium having stored thereon computer-executable instructions for generating at least one magnetic resonance imaging (MRI) temperature profile of at least one portion of at least one patient, wherein, when a computer arrangement executes the instructions, the computer arrangement is configured to perform procedures comprising:
receiving first imaging information related to an MRI scan of the at least one portion of the at least one patient;
generating second imaging information by segmenting the first imaging information into a plurality of layers; and
generating the at least one MRI temperature profile by applying at least one bioheat equation to the second imaging information.

2. The computer-accessible medium of claim 1, wherein the first imaging information is based on a point-by-point MRI scan of the at least one patient.

3. The computer-accessible medium of claim 1, wherein the first imaging information is a Digital Imaging and Communications in Medicine data set produced by an MRI apparatus.

4. The computer-accessible medium of claim 1, wherein the layers include at least three layers, and wherein a first layer of the layers is a high water content, a second layer of the layers is a low water content, and a third layer of the layers is an air content.

5. The computer-accessible medium of claim 4, wherein the layers include at least four layers, and wherein a fourth later of the layers is the at least one portion of a lung.

6. The computer-accessible medium of claim 1, wherein the computer arrangement is further configured to assign at least one of a conductivity or a permittivity to the second imaging information associated with the at least one portion, and wherein the at least one bioheat equation is based on the at least one of the conductivity or the permittivity.

7. The computer-accessible medium of claim 6, wherein the computer arrangement is further configured to assign the at least one of the conductivity or the permittivity based on at least one thermal property of the at least one portion.

8. The computer-accessible medium of claim 7, wherein the at least one thermal property includes at last one of (i) a specific absorption rate, (ii) a specific heat, (iii) a density, or (iv) a perfusion.

9. The computer-accessible medium of claim 1, wherein the at least one MRI temperature profile includes (i) areas to heat for the at least one portion of the at least one patient and (ii) magnitude assignments for the at least one portion of the at least one patient.

10. The computer-accessible medium of claim 1, wherein the at least one bioheat equation is based on a specific absorption rate of the at least one portion of the at least one patient.

11. A method for generating at least one magnetic resonance imaging (MRI) temperature profile of at least one portion of at least one patient, comprising:
receiving first imaging information related to an MRI scan of the at least one portion of the at least one patient;
generating second imaging information by segmenting the first imaging information into a plurality of layers; and
using a computer hardware arrangement, generating the at least one MRI temperature profile by applying at least one bioheat equation to the second imaging information.

12-15. (canceled)

16. The method of claim 11, further comprising assigning at least one of a conductivity or a permittivity to the second imaging information associated with the at least one portion, wherein the at least one bioheat equation is based on the at least one of the conductivity or the permittivity.

17. The method of claim 16, wherein the assigning of the at least one of the conductivity or the permittivity is based on at least one thermal property of the at least one portion.

18. The method of claim 17, wherein the at least one thermal property includes at least one of (i) a specific absorption rate, (ii) a specific heat, (iii) a density, or (iv) a perfusion.

19-20. (canceled)

21. A system for generating at least one magnetic resonance imaging (MRI) temperature profile of at least one portion of at least one patient, comprising:
a computer hardware arrangement configured to:
receive first imaging information related to an MRI scan of the at least one portion of the at least one patient;
generate second imaging information by segmenting the first imaging information into a plurality of layers; and
generate the at least one MRI temperature profile by applying at least one bioheat equation to the second imaging information.

22-30. (canceled)

31. A non-transitory computer-accessible medium having stored thereon computer-executable instructions for generating a magnetic resonance image (MRI) of at least one portion of at least one patient, wherein, when a computer arrangement executes the instructions, the computer arrangement is configured to perform procedures comprising:
performing a first MRI scan of the at least one portion of the at least one patient using an MRI apparatus;
generating first imaging information based on the first MRI scan;
transmitting the first imaging information over a network;
receiving, over the network, second imaging information related to at least one MRI temperature profile of the at least one portion that is based on at least one bioheat equation;
modifying parameters of the MRI apparatus based on the second imaging information;
performing a second MRI scan of the at least one portion using the modified parameters.

32. The computer-accessible medium of claim 31, wherein the modified parameters include (i) local areas of heating for the at least one portion, and (ii) magnitude assignments for the at least one portion.

33. The computer-accessible medium of claim 31, wherein the first imaging information is based on a point-by-point MRI scan of the at least one patient.

34. The computer-accessible medium of claim 31, wherein the at least one bioheat equation is based on a specific absorption rate of the at least one portion.

35-42. (canceled)
